(19) United States
(12) Patent Application Publication
(10) Pub. No.: US 2005/0214941 A1
(43) Pub. Date: Sep. 29, 2005

(54) Expansion of neural stem cells with LIF

(75) Inventors: Smita Bhonsale, Ellicott City, MD (US); Padmavathy Vanguri, Cockeysville, MD (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Theradigm, Inc.

(21) Appl. No.: 11/083,231

(22) Filed: Mar. 16, 2005

Related U.S. Application Data
(60) Provisional application No. 60/553,724, filed on Mar. 16, 2004.

(51) Int. Cl. C12N 5/08

(52) U.S. Cl. 435/368

(57) ABSTRACT

The present invention encompasses methods and compositions for enhancing the growth of neural stem cells (NSCs).

### CROSS-REFERENCE TO RELATED APPLICATIONS

This application claims priority pursuant to 35 U.S.C. § 119(e) to U.S. Provisional Application No. 60/553,724, filed Mar. 16, 2004, which is hereby incorporated by reference in entirely herein.

### BACKGROUND OF THE INVENTION

Stem cells are self-renewing multipotent progenitors with the broadest developmental potential in a given tissue at a given time (Morrison et al. 1997 Cell 88:287-298). A great deal of interest has recently been attracted by studies of stem cells in the nervous system, not only because of their importance for understanding neural development but also for their therapeutic potential in the treatment of neurodegenerative diseases.

During development of the central nervous system “CNS”, multipotent precursor cells, also known as neural stem cells, proliferate, giving rise to transiently dividing progenitor cells that eventually differentiate into the cell types that compose the adult brain. Stem cells (from other tissues) have classically been defined as having the ability to self-renew (i.e., form more stem cells), to proliferate, and to differentiate into multiple different phenotypic lineages. In the case of neural stem cells, this includes neurons, astrocytes and oligodendrocytes. For example, Potten and Loeffler (1990, Development 110:1001-20) characterized stem cells as undifferentiated cells capable of proliferating, self-maintenance, production of a large number of differentiated functional progeny and regenerating a tissue after injury.

Neural stem cells (NSCs) have been isolated from several mammalian species, including mice, rats, pigs and humans (WO 93/01275, WO 94/09119, WO 94/10292, WO 94/16718; Cattaneo et al., 1996, Mol. Brain Res. 42:161-66). Human CNS neural stem cells, like their rodent homologs, when maintained in a mitogen-containing (typically epidermal growth factor (EGF) or EGF plus basic fibroblast growth factor (bFGF)) and serum-free culture medium, grow in suspension culture to form aggregates of cells known as “neurospheres”. It has been observed that human neural stem cells have doubling rates of about 30 days (Cattaneo et al., 1996, Mol Brain Res. 42:161-66). Others have shown doubling times ranging from 7-14 days in the presence of FGF and EGF (Vescovi et al., 1999 Brain Pathol. 9:569-98). Upon removal of the mitogen(s), the stem cells can differentiate into neurons, astrocytes and oligodendrocytes.

To improve the growth rate of human fetal brain stem cells, several different methods and growth factors have been used by a number of different investigators during the last decade. It has been demonstrated that basic fibroblast growth factor (bFGF) and epidermal growth factor (EGF) are needed for expansion and maintenance of human fetal neural stem cells (hNSCs). These human NSC cultures are normally grown as free floating clusters of cells (neurospheres), but the neurospheres cannot proliferate indefinitely in the presence of bFGF and EGF alone. Addition of leukemia inhibitory factor (LIF) was shown to enhance proliferation of NSCs by decreasing doubling times to 7 days (Carpenter et al, 1999, Exp. Neurol. 158:265-278) and 4.5 days (Wright et al., 2003, J. Neurochem. 86:179-795).

Human fetal brain stem cells are considered to be attractive candidates for stem cell transplantation for regeneration of damaged tissues. The transplantation of cells between genetically disparate individuals invariably is associated with the risk of graft rejection by the host. Nearly all cells express products of the major histocompatibility complex, MHC class I molecules. Further, many cell types can be induced to express MHC class II molecules when exposed to inflammatory cytokines. Rejection of allografts is mediated primarily by T cells of both the CD4 and CD8 subclasses (Rosenberg et al. 1992 Annu. Rev. Immunol. 10:333). Alloreactive CD4 T cells produce cytokines that exacerbate the cytolytic CD8 response to an alloantigen. Within these subclasses, competing subpopulations of cells develop after antigen stimulation that are characterized by the cytokines they produce. Th1 cells, which produce IL-2 and IFN-γ, are primarily involved in allograft rejection (Mossmann et al., 1989 Annu. Rev. Immunol. 7:145). Th2 cells, which produce IL-4 and IL-10, can down-regulate Th1 responses through IL-10 (Fiorentino et al. 1989 J. Exp. Med. 170:2081). Indeed, much effort has been expended to divert undesirable Th1 responses toward the Th2 pathway. Undesirable alloreactive T cell responses against a transplant in patients are typically treated with immunosuppressive drugs such as prednisone, azathioprine, and cyclosporine A. Unfortunately, these drugs generally need to be administered for the life of the patient and they have a multitude of dangerous side effects including generalized immunosuppression.

Neural stem cells have been shown to express low or negligible levels of MHC class I and/or class II antigens (McLaren et al. 2001 J. Neuroimmunol. 112:35), and cells cultured according to McLaren et al. are usually rejected after implantation into allogeneic recipients unless immunosuppressive drugs are used. Rejection may be initiated after MHC molecules are up-regulated on cell membranes after exposure to inflammatory cytokines of the IFN family.

There remains a need to increase the rate of proliferation of neural stem cell cultures. There also remains a need to increase the number of neurons in the differentiated cell population. There further remains a need to improve the viability of neural stem cell grafts upon implantation into a host. Thus, there is a strong need for standardization of culture conditions for maximizing the proliferation and multipotentiality of NSCs. The present invention satisfies this need.

### BRIEF SUMMARY OF THE INVENTION

The invention comprises compositions and methods for culturing a Neural Stem Cell (NSC) on a coated surface to enhance the proliferation rate without losing the capacity to differentiate.

The invention includes a composition comprising an in vitro adherent culture comprising an NSC, wherein the NSC cell proliferates in the presence of LIF while maintaining multipotentiality of the NSC.

In one aspect, the NSC adheres to a surface coated with polyomithine and fibronectin.

In another aspect, the NSC is derived from a human.

In yet another aspect, exogenous genetic material has been introduced into the NSC.

The invention also includes a method for the in vitro expansion and maintenance of the multipotentiality of an NSC.

In one aspect, the method comprises culturing an NSC as an adherent cell population on a coated surface in the presence of LIF.

In another aspect, the method comprises culturing an NSC on a surface coated with polyornithine and fibronectin.

In yet another aspect, the method comprises culturing a human NSC.

In a further aspect, exogenous genetic material has been introduced into the NSC.

The invention includes a method for the in vitro expansion and maintenance of the multipotentiality of an NSC, the method comprises culturing an NSC as an adherent population on a coated surface in the presence of LIF, wherein the expression of MHC class II molecule in said NSC is regulated by the method.

The invention also includes a method for the in vitro expansion and maintenance of the multipotentiality of an NSC, wherein the expression of MHC class II molecule is reduced in said NSC when compared to an otherwise identical NSC cultured in the continuous presence of LIF.

In one aspect, the method includes culturing an NSC as an adherent population on a coated surface in the presence of LIF for a period of time, then removing LIF from the culture, and culturing said NSC as an adherent population on a coated surface in the absence of LIF for a period of time.

In a another aspect, NSCs are cultured in the presence of LIF for about 7 days.

In yet another aspect, NSCs are cultured in the absence of LIF for about 7 days.

In a further aspect, NSCs exhibits a doubling rate of about 28-36 hours following the culturing of the NSCs in the presence of LIF for a period of time and then in the absence of LIF for a period of time.

The invention includes an NSC prepared by a method of culturing said NSC as an adherent population on a coated surface in the presence of LIF for a period of time, then removing LIF from the culture, and culturing said NSC as an adherent population on a coated surface in the absence of LIF for a period of time.

In one aspect, the NSC exhibits a doubling rate of about 28-36 hours.

In another aspect, the NSC exhibits a reduced level of MHC class II molecule expression compared to the level of MHC class II molecule expression on an otherwise identical NSC cultured in the continuous presence of LIF.

In yet another aspect, the NSC is derived from a human.

In a further aspect, exogenous genetic material has been introduced into the NSC.

The invention includes a method of treating a human patient having a disease, disorder or condition of the central nervous system.

In one aspect, the method includes obtaining an isolated NSC, culturing the NSC as an adherent population on a coated surface in the presence of LIF for a period of time, removing LIF from the culture, culturing the NSC as an adherent population on a coated surface in the absence of LIF for a period of time, and administering the cultured NSC to a patient in need thereof.

In another aspect, the disease, disorder or condition of the central nervous system is selected from the group consisting of a genetic disease, brain trauma, Huntington's disease, Alzheimer's disease, Parkinson's disease, spinal cord injury, stroke, multiple sclerosis, cancer, CNS lysosomal storage diseases and head trauma, epilepsy.

In yet another aspect, the disease, disorder or condition is injury to the tissue or cells of the central nervous system.

In a further aspect, the disease, disorder or condition is a brain tumor.

In one aspect, cultured NSCs administered to the central nervous system remain present and/or replicate in the central nervous system.

In a further aspect, the NSC is cultured in vitro in a differentiation medium prior to administering the NSC into a patient in need thereof.

In yet another aspect, the NSC is genetically modified prior to administering the NSC into a patient in need thereof.

The invention includes a composition comprising an isolated NSC and a biologically compatible lattice.

In one aspect, the NSC is cultured as an adherent population on a biologically compatible lattice in the presence of LIF for a period of time and in the absence of LIF for a period of time.

In another aspect, the lattice comprises a polymeric material.

In a further aspect, the polymeric material is formed of polymer fibers as a mesh or sponge.

In yet another aspect, the polymeric material comprises monomers selected from the group of monomers consisting of glycolic acid, lactic acid, propyl fumarate, caprolactone, hyaluronan, hyaluronic acid and combinations thereof.

In another aspect, the polymeric material comprises proteins, polysaccharides, polyhydroxy acids, polyorthoesters, polyanhydrides, polyphosphazenes, synthetic polymers or combinations thereof.

In a further aspect, the polymeric material is a hydrogel formed by crosslinking of a polymer suspension having the cells dispersed therein.

In one aspect, the biologically compatible lattice is further coated with polyomithine.

In another aspect, the biologically compatible lattice is further coated with fibronectin.

In yet another aspect, the biologically compatible lattice is further coated with polyomithine and fibronectin.

The invention includes a method for the in vitro expansion and maintenance of the multipotentiality of a neural stem cell (NSC), the method comprising culturing a NSC as an adherent population on a biologically compatible lattice in the presence of LIF for a period of time and in the absence of LIF for a period of time.

In one aspect, the biologically compatible lattice is further coated with polyornithine.

In another aspect, the biologically compatible lattice is further coated with fibronectin.

In yet another aspect, the biologically compatible lattice is further coated with polyomithine and fibronectin.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A through 1D, is a series of images depicting undifferentiated human neural stem cell cultures. FIG. 1A depicts THD-hWB-015 cells cultured in the absence of Leukemia Inhibitory Factor (LIF). FIG. 1B depicts THD-hWB-015 cells cultured in the presence of LIF. FIGS. 1C and 1D depict THD-hFB-17 cells cultured in the presence (FIG. 1D) and absence (FIG. 1C) of LIF, respectively. In all cases the NSCs were grown on coated dishes.

FIG. 2, comprising FIGS. 2A and 2B, is a set of graphs depicting the cumulative fold expansion of NSCs cultured in the absence and presence of HLIF. Parallel cultures are maintained over 200 days in EGF+bFGF in the presence or absence of human Leukemia Inhibitory Factor (hLIF). Cultures grown in the absence of LIF demonstrated significantly lower expansion rate than cells grown in the presence of LIF. FIG. 2A depicts the growth curves of THD-hWB-015 cells and FIG. 2B depicts the growth curves of THD-hFB-017 cells. When grown on coated dishes, the doubling time for THD-hWB-015 in the presence of LIF was 24-28 hours compared with 70-74 hours in the absence of LIF. Doubling time for THD-hFB-017 was 40-43 hours in the presence of LIF compared with 90-94 hours in the absence of LIF.

FIG. 3, comprising FIGS. 3A through 3D, is a series of images depicting BrdU incorporation into undifferentiated NSCs. BrdU incorporation was examined in two different cultures in the absence (FIGS. 3A and 3C) and presence (FIGS. 3B and 3D) of hLIF. Incorporation of BrdU into the cells represents active proliferation. Cells were plated on a coated chamber slide in complete growth medium +/−hLIF. 20 μM BrdU was added for 24 hrs prior to fixing the cells, and the cells were subsequently stained with anti-BrdU antibodies and secondary antibodies conjugated with Alexa 488. There were on average 20-30% cells that were BrdU positive in the absence of LIF, while 70-75% cells were positive for BrdU in the presence of LIF. FIGS. 3A and 3B depict THD-hWB-015 cells in the presence (FIG. 3B) and absence (FIG. 3A) of LIF. FIGS. 3C and 3D depict THD-hFB-17 cells in the presence (FIG. 3D) and absence (FIG. 3C) of LIF.

FIG. 4, comprising FIGS. 4A through 4D, is a series of images depicting nestin expression (marker to identify undifferentiated NSCs) in undifferentiated cells plated on coated chamber slides. FIGS. 4A and 4B depict THD-hWB-015 cells in the presence (FIG. 4B) and absence (FIG. 4A) of LIF. FIGS. 4C and 4D depict THD-hFB-17 cells in the presence (FIG. 4D) and absence (FIG. 4C) of LIF. In every case, 94-99% of the cells were nestin positive. In FIG. 4A (THD-hWB-015 without LIF), 86% of the cells were nestin only positive, 8-10% of the cells were positive for nestin and glial fibrillary acidic protein (GFAP is a marker for astrocytes), and 3-4% were positive for GFAP only. In FIG. 4B (with LIF), 98% of the cells were both GFAP and nestin positive while only 1-2% of the cells were GFAP positive only.

FIG. 5, comprising FIGS. 5A through 5D, is a series of images depicting in vitro differentiation of human NSC cultures (THD-hWB-015 and THD-hFB-017) grown in the presence or absence of hLIF. FIGS. 5B and 5D demonstrate that the presence of hLIF in the growth medium did not affect multipotency of these cultures. Cultures were differentiated as described elsewhere herein for 14 days.

FIG. 6, comprising FIGS. 6A through 6H, is a series of FACS analysis graphs depicting the phenotype of NSCs (NSC line designated THD-hWB-015 (P13)) following culturing in a medium supplemented with bFGF and EGF for 14 days. FIGS. 6A through 6H depict the profile of CD45, CD86, CD14, CD133, CD80, CD34, MHC Class II molecule and MHC Class I molecule, respectively.

FIG. 7, comprising FIGS. 7A through 7H, is a series of FACS analysis graphs depicting the phenotype of NSCs (NSC line designated THD-hWB-015 (P13)) following culturing in a medium supplemented with bFGF, EGF, and LIF for 14 days. FIGS. 7A through 7H depict the profile of CD45, CD86, CD14, CD133, CD80, CD34, MHC Class II molecule and MHC Class I molecule, respectively.

FIG. 8, comprising FIGS. 8A through 8H, is a series of FACS analysis graphs depicting the phenotype of NSCs (NSC line designated THD-hWB-015 (P13)) following culturing in a medium supplemented with bFGF, EGF, and LIF for 7 days, and then cultured in an otherwise identical medium supplemented with bFGF and EGF, but in the absence of LIF. FIGS. 8A through 8H depict the profile of CD45, CD86, CD14, CD133, CD80, CD34, MHC Class II molecule and MHC Class I molecule, respectively.

FIG. 9, comprising FIG. 9A through 9D, is a series of FACS analysis graphs depicting the phenotype of NSCs (NSC line designated THD-hWB-015) following culturing under four different growth conditions as follows: uncoated flasks (FIG. 9A); uncoated flasks in the presence of LIF (FIG. 9B); coated flasks (FIG. 9C); and coated flasks in the presence of LIF (FIG. 9D). For each growth condition, the cultured cells were analyzed for the expression of CD56, CD 184, CD 117, and CD133, respectively.

### DETAILED DESCRIPTION

In prior art methods, NSCs are typically cultured in the presence of growth factors such as basic fibroblast growth factor (bFGF) and epidermal growth factor (EGF) as free floating clusters of cells (neurospheres). According to the methods of the present invention, NSCs are cultured as an adherent population. Preferably, the adherent culture is grown on a coated surface, and more preferably, the culture medium is supplemented with leukemia inhibitory factor (LIF). One skilled in the art would recognize based upon the present disclosure that the surface can be coated with an extracellular matrix component. The extracellular matrix component can include but is not limited to, polyornithine and/or fibronectin. Preferably, the extracellular matrix component is bovine fibronectin or porcine fibronectin. More preferably, the extracellular matrix component is human fibronectin. Further, one skilled in the art would recognize that other growth factors known in the art can be used to enhance proliferation of NSCs.

The present invention comprises methods and compositions for inducing or enhancing proliferation of neural stem cells (NSCs) while preserving their multipotentiality. The present invention also relates to the discovery that the expression of MHC molecules by NSCs can be modulated by culturing NSCs according to the methods disclosed herein. The disclosure presented herein demonstrates that in addition to enhancing the proliferation of NSCs while preserving their multipotential capacities, culturing NSCs as an adherent cell population in the presence of LIF, modulates the upregulation and/or induction of MHC molecule expression by NSCs compared with the expression of MHC molecules by NSCs cultured using standard methods known in the art. That is, the present invention provides a method of culturing NSCs in a manner that provides additional benefits over the standard methods used for enhancing proliferation of NSCs in culture, in that the basis for rejection of these cells in a recipient can be controlled.

The present invention also relates to the discovery that the expression of major histocompatibility complex class II (MHC class II) molecules by NSCs is regulated by LIF. That is, the expression of MHC class II molecules by NSCs can be regulated using the culturing methods of the present invention, for example growing the NSCs as an adherent population in the presence of LIF.

In a further embodiment of the present invention, the expression of MHC II molecule by NSCs can be regulated using the method comprising growing the NSCs in the presence of LIF for a period of time and then growing the NSCs in the absence of LIF for a period of time. In a preferred method, the cells are grown in the presence of LIF for about 7 days and subsequently grown in the absence of LIF for about another 7 days.

In a further embodiment of the present invention, the expression of MHC class II molecules by NSCs can be reduced using the method comprising growing the NSCs in the presence of LIF for a period of time and then growing the NSCs in the absence of LIF for a period of time, as compared with growing the NSCs only in the presence of LIF. In a preferred method, the cells are grown in the presence of LIF for about 7 days and subsequently grown in the absence of LIF for about another 7 days.

The NSC culture/expansion method of the invention, as used herein, refers to the method of enhancing proliferation of NSCs. Preferably, the method of enhancing proliferation of NSCs encompasses culturing an adherent population of NSCs in the presence of LIF, wherein the NSCs retain their multipotentiality (their capacity to differentiate into one of various cell types, such as neurons, astrocytes, oligodendrocytes and the like).

In a further embodiment of the present invention, NSCs expanded using the methods of the present invention retain their ability to differentiate to a greater extent (i.e., in greater proportion) into neurons than do NSCs expanded or cultured using prior art methods.

The NSC culture/expansion methods described herein solve an essential problem for the generation of NSCs for use as a treatment of human diseases. That is, prior to the disclosure provided herein, NSCs were difficult to isolate and expand in culture (i.e., it was difficult to induce them to proliferate in sufficient number for therapeutic purposes). The disclosure provided herein demonstrates that NSCs can be grown and isolated in large numbers for therapeutic uses and this distinguishes the present invention from prior art disclosures.

The neural stem cells of the present invention may be proliferated in an adherent culture. When the neural stem cells of this invention are proliferating, nestin antibody can be used as a marker to identify undifferentiated cells and distinguish them from differentiated cells.

When the cells of the present invention were differentiated, most of the cells lost their nestin positive immunoreactivity. Further, antibodies specific for various neuronal or glial proteins may be employed to identify the phenotypic properties of the differentiated cells. Neurons may be identified using antibodies to neuron specific neurofilament, Tau, beta-tubulin, or other known neuronal markers. Astrocytes may be identified using antibodies to glial fibrillary acidic protein “GFAP”, or other known astrocytic markers. Oligodendrocytes may be identified using antibodies to galactocerebroside, 04, myelin basic protein “MBP” or other known oligodendrocytic markers. Glial cells in general may be identified by staining with antibodies, such as the M2 antibody, or other known glial markers.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

“Allogeneic” refers to a graft derived from a different animal of the same species. As used herein, the term “autologous” is meant to refer to any material derived from the same individual to which it is re-introduced.

As used herein, the term “biocompatible lattice,” is meant to refer to a substrate that can facilitate formation into three-dimensional structures conducive for tissue development. Thus, for example, cells can be cultured or seeded onto such a biocompatible lattice, such as one that includes extracellular matrix material, synthetic polymers, cytokines, growth factors, etc. The lattice can be molded into desired shapes for facilitating the development of tissue types. Also, at least at an early stage during culturing of the cells, the medium and/or substrate is supplemented with factors (i.e., growth factors, cytokines, extracellular matrix material, etc.) that facilitate the development of appropriate tissue types and structures.

As used herein, “central nervous system” should be construed to include brain and/or the spinal cord of a mammal. The term may also include the eye and optic nerve in some instances.

The term “coated” is used herein to refer to a surface that has been treated with an extracellular component. The coated surface provides a surface on which cells may adhere. Examples of an extracellular component include but not limited to fibronectin, laminin, poly-D-lysine and poly-L-lysine.

As used herein, the term “disease, disorder or condition of the central nervous system” is meant to refer to a disease, disorder or a condition which is caused by a genetic mutation in a gene that is expressed by cells of the central nervous system such that one of the effects of such a mutation is manifested by abnormal structure and/or function of the central nervous system, such as, for example, neurodegenerative disease or primary tumor formation. Such genetic defects may be the result of a mutated, non-functional or under-expressed gene in a cell of the central nervous system. The term should also be construed to encompass other pathologies in the central nervous system which are not the result of a genetic defect per se in cells of the central nervous system, but rather are the result of infiltration of the central nervous system by cells which do not originate in the central nervous system, for example, metastatic tumor formation in the central nervous system. The term should also be construed to include trauma to the central nervous system induced by direct injury to the tissues of the central nervous system.

“Differentiated” is used herein to refer to a cell that has achieved a terminal state of maturation such that the cell has developed fully and demonstrates biological specialization and/or adaptation to a specific environment and/or function. Typically, a differentiated cell is characterized by expression of genes that encode differentiated associated proteins in that cell. For example expression of myelin proteins and formation of a myelin sheath in a glial cell is a typical example of a terminally differentiated glial cell. When a cell is said to be “differentiating,” as that term is used herein, the cell is in the process of being differentiated.

“Differentiation medium” is used herein to refer to a cell growth medium comprising an additive or a lack of an additive such that a stem cell, embryonic stem cell, ES-like cell, neurosphere, NSC or other such progenitor cell, that is not fully differentiated, when incubated in the medium, develops into a cell with some or all of the characteristics of a differentiated cell.

“Expandability” is used herein to refer to the capacity of a cell to proliferate for example to expand in number, or in the case of a cell population, to undergo population doublings.

“Graft” refers to a cell, tissue, organ or otherwise any biological compatible lattice for transplantation.

As used herein, the term “growth medium” is meant to refer to a culture medium that promotes growth of cells. A growth medium will generally contain animal serum. In some instances, the growth medium may not contain animal serum but may contain mitogens.

“Leukemia Inhibitory Factor” (LIF) is used herein to refer to a 22 kDa protein member of the interleukin-6 cytokine family that has numerous biological functions. LIF has been demonstrated to have the capacity to induce terminal differentiation in leukemic cells, induce hematopoietic differentiation in normal and myeloid leukemia cells, and to stimulate acute-phase protein synthesis in hepatocytes. LIF has also been shown herein to enhance proliferation of NSCs in an undifferentiated state while maintaining the multipotentiality of the NSCs.

As used herein, the term “LIF+/− regimen” refers to a culturing method of growing a cell in the presence of LIF for a period of time and then culturing the cell in the absence of LIF for another period of time.

As used herein, the term “modulate” is meant to refer to any change in biological state, i.e. increasing, decreasing, and the like. “Modulate MHC class molecule express” is used herein to refer to any change in the expression of MHC class molecules expressed by a cell. Based on the disclosure herein, it was observed that the expression of MHC class II molecules by NSCs was closely regulated by LIF. In the presence of LIF, MHC class II molecules were displayed on the NSCs. It was also observed that the expression of MHC class II molecules by NSCs was reduced when the cells were grown according to a regimen of growing the cells in the presence of LIF for a period of time and then growing the cells in the absence of LIF for another period of time as compared with expression of MHC class II molecules in cells grown in the presence of LIF for a period of time.

As used herein, the term “multipotential” or “multipotentiality” is meant to refer to the capability of a stem cell of the central nervous system to differentiate into more than one type of cell. For example a multipotential stem cell of the central nervous system is capable of differentiating into cells including but not limited to neurons, astrocytes and oligodendrocytes.

“Neurosphere” is used herein to refer to a neural stem cell/progenitor cell wherein nestin expression can be detected, including, inter alia, by immunostaining to detect nestin protein in the cell. Neurospheres are aggregates of proliferating neural stem/progenitor cells, and the formation of neurosphere is a characteristic feature of neural stem cells in in vitro culture.

“Neural stem cell” is used herein to refer to undifferentiated, multipotent, self-renewing neural cell. A neural stem cell is a clonogenic multipotent stem cell which is able to divide and, under appropriate conditions, has self-renewal capability and can terminally differentiate into neurons, astrocytes, and oligodendrocytes. Hence, the neural stem cell is “multipotent” because stem cell progeny have multiple differentiation pathways. A neural stem cell is capable of self maintenance, meaning that with each cell division, one daughter cell will also be, on average, a stem cell.

“Neural cell” is used herein to refer to a cell that exhibits a morphology, a function, and a phenotypic characteristic similar to that of glial cells and neurons derived from the central nervous system and/or the peripheral nervous system.

“Neuron-like cell” is used herein to refer to a cell that exhibits a morphology similar to that of a neuron and detectably expresses a neuron-specific marker, such as, but not limited to, MAP2, neurofilament 200 kDa, neurofilament-L, neurofilament-M, synaptophysin, β-tubulin III (TUJ1), Tau, NeuN, a neurofilament protein, and a synaptic protein.

“Astrocyte-like cell” is used herein to refer to a cell that exhibits a phenotype similar to that of an astrocyte and which expresses the astrocyte-specific marker, such as, but not limited to, GFAP.

“Oligodendrocyte-like cell” is used herein to refer to a cell that exhibits a phenotype similar to that of an oligodendrocyte and which expresses the oligodendrocyte-specific marker, such as, but not limited to, O-4.

“Progression of or through the cell cycle” is used herein to refer to the process by which a cell prepares for and/or enters mitosis and/or meiosis. Progression through the cell cycle includes progression through the G1 phase, the S phase, the G2 phase, and the M-phase.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cells, and the like.

“Transplant” refers to a biocompatible lattice or a donor tissue, organ or cell, to be transplanted.

As used herein, a “therapeutically effective amount” is the amount of cells which is sufficient to provide a beneficial effect to the subject to which the cells are administered.

“Xenogeneic” refers to a graft derived from an animal of a different species.

As used herein, a “substantially purified” cell is a cell that is essentially free of other cell types. Thus, a substantially purified cell refers to a cell which has been purified from other cell types with which it is normally associated in its naturally occurring state. As used herein, the term “exogenous” refers to any material introduced from or produced outside an organism, cell, or system.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The phrase “under transcriptional control” or “operatively linked” as used herein means that the promoter is in the correct location and orientation in relation to the polynucleotides to control RNA polymerase initiation and expression of the polynucleotides.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (i.e., naked or contained in liposomes) and viruses that incorporate the recombinant polynucleotide.

### DESCRIPTION

The present invention includes a method of enhancing the proliferation while maintaining the multipotential capacity of NSCs. Preferably, the NSCs are derived from a mammal, more preferably the NSCs are derived from a human. The method comprises isolating NSCs using methods well known in the art and culturing NSCs on a coated surface maintained as an adherent culture that expands into adherent and/or non-adherent neurospheres cultures. Preferably, the isolated NSCs are cultured as an adherent culture in the present of LIF. More preferably, the isolated NSCs are cultured as an adherent culture and expands into an adherent culture in the presence of LIF.

The invention relates to the discovery that the expandability of NSCs (the capacity of NSCs to replicate themselves multiple times) can be increased by the combination of growing NSCs as an adherent population in the presence of LIF. In a further embodiment of the present invention, an NSC adherent population is cultured on a coated surface in the presence of LIF to enhance their proliferation rate without losing their capacity to differentiate.

The present invention also relates to the discovery that the expression of MHC molecules by NSCs can be modulated by culturing NSCs according to the methods disclosed herein. The disclosure presented herein demonstrates that in addition to enhancing the proliferation of NSCs while preserving their multipotential capacities, culturing NSCs as an adherent cell population in the presence of LIF modulates the upregulation and/or induction of MHC molecule expression by NSCs compared with the expression of MHC molecules by NSCs cultured using standard methods known in the art. As such, the present invention provides a method of culturing NSCs in a manner that provides additional benefits over the standard methods used for enhancing proliferation of NSCs in culture.

Isolation of NSCs

NSCs can be obtained from the central nervous system of a mammal, preferably a human. These cells can be obtained from a variety of tissues including but not limited to, fore brain, hind brain, whole brain and spinal cord. NSCs can be isolated and cultured using the methods detailed elsewhere herein or using methods known in the art, for example using methods disclosed in U.S. Pat. No. 5,958,767 hereby incorporated by reference herein in its entirety. Other methods for the isolation of NSCs are well known in the art, and can readily be employed by the skilled artisan, including methods to be developed in the future. For example, NSCs have been isolated from several mammalian species, including mice, rats, pigs and humans. See, i.e., WO 93/01275, WO 94/09119, WO 94/10292, WO 94/16718 and Cattaneo et al. (1996 Mol. Brain Res. 42:161-66), all herein incorporated by reference. The present invention is in no way limited to these or any other methods of obtaining a cell of interest.

Any suitable tissue source may be used to derive the NSCs of this invention. NSCs can be induced to proliferate and differentiate either by culturing the cells in suspension or on an adherent substrate. See, i.e., U.S. Pat. No. 5,750,376 and U.S. Pat. No. 5,753,506 (both incorporated herein by reference in their entirety), and medium described therein. Both allografts and autografts are contemplated for transplantation purposes.

NSCs can be isolated from many different types of tissues, for example, from donor tissue by dissociation of individual cells from the connecting extracellular matrix of the tissue, or from commercial sources of NSCs. In one example, tissue from brain is removed using sterile procedures, and the cells are dissociated using any method known in the art including treatment with enzymes such as trypsin, collagenase and the like, or by using physical methods of dissociation such as mincing or treatment with a blunt instrument. Dissociation of neural cells, and other multipotent stem cells, can be carried out in a sterile tissue culture medium. Dissociated cells are centrifuged at low speed, between 200 and 2000 rpm, usually between 400 and 800 rpm, the suspension medium is aspirated, and the cells are then resuspended in culture medium.

Treatment of NSCs

The invention comprises methods and compositions for the treatment of NSCs to enhance their proliferation rate without losing their capacity to differentiate. While not wishing to be bound by any particular theory, it is believed that the treatment of the NSCs with a defined medium supplemented with LIF, in a 2-dimensional or 3-dimensional biocompatible lattice, enhances the proliferation rate of NSCs while maintaining the multipotential capacity of NSCs.

In one embodiment of the present invention, the cells are cultured on a surface coated with polyomithine and fibronectin. However, the present invention should not be construed to only include culturing the cells solely on the presence of these compounds. Rather, the present invention should encompass any biocompatible material that can be used to culture NSCs as an adherent culture.

The invention also comprises culturing NSCs in a defined medium in a 2-dimensional or 3-dimensional biocompatible lattice. The use of a biocompatible lattice facilitates in vivo tissue engineering by supporting and/or directing the fate of the implanted cells. For example, the invention can facilitate the regeneration of brain tissue by culturing the inventive NSCs under conditions suitable for them to expand and divide to form a desired structure. In some applications, this is accomplished by transferring them to an animal typically at a site at which the new matter is desired.

In another embodiment, the cells can be induced to differentiate and expand into a desired tissue in vitro. In such an application, the cells are cultured on substrates that facilitate formation into three-dimensional structures conducive for tissue development. Thus, for example, the cells can be cultured or seeded onto a bio-compatible lattice, such as one that includes extracellular matrix material, synthetic polymers, cytokines, growth factors, and the like. Such a lattice can be molded into desired shapes for facilitating the development of tissue types. Also, at least at an early stage during such culturing, the medium and/or substrate is supplemented with factors (i.e., growth factors, cytokines, extracellular matrix material, and the like) that facilitate the development of appropriate tissue types and structures. Indeed, in some embodiments, it is desired to co-culture the cells with mature cells of the respective tissue type, or precursors thereof, or to expose the cells to the respective medium, as discussed herein.

To facilitate the use of the NSC of the present invention for producing such a tissue, the invention provides a composition including the inventive cells (and populations) and a biologically compatible lattice. Typically, the lattice is formed from polymeric material, having fibers as a mesh or sponge, typically with spaces on the order of between about 100 μm and about 300 μm. Such a structure provides sufficient area on which the cells can grow and proliferate. Preferably, the lattice is biodegradable over time, so that it will be absorbed into the animal matter as it develops. Suitable polymeric lattices, thus, can be formed from monomers such as glycolic acid, lactic acid, propyl fumarate, caprolactone, hyaluronan, hyaluronic acid, and the like. Other lattices can include proteins, polysaccharides, polyhydroxy acids, polyorthoesthers, polyanhydrides, polyphosphazenes, or synthetic polymers (particularly biodegradable polymers). Of course, a suitable polymer for forming such lattice can include more than one monomers (i.e., combinations of the indicated monomers). Also, the lattice can also include hormones, such as growth factors, cytokines, and morphogens (i.e., retinoic acid, aracadonic acid, and the like), desired extracellular matrix molecules (i.e., polyornithine, fibronectin, laminin, collagen, and the like), or other materials (i.e., DNA, viruses, other cell types, and the like) as desired.

In another embodiment, the invention provides a lattice composition comprising NSCs of the present invention and mature/differentiated cells of a desired phenotype thereof, particularly to potentate the induction of the inventive NSCs to differentiate appropriately within the lattice (i.e., as an effect of co-culturing such cells within the lattice).

Without wishing to be bound by any particular theory, one benefit of culturing the cells as an adherent cell population is to obtain a more homogenous cell population than that possible when the cells are grown as a free floating cluster of cells known as neurospheres. In addition, an adherent population of cells provides a means for the cell population to be exposed more uniformly to factors (i.e. growth factors, trophic factors and the like) present in the culture medium.

In another embodiment of the present invention, the cells cultured as an adherent cell population on a coated surface in the presence of LIF were observed to have a heightened proliferation rate without losing their capacity to differentiate into cell types including, but not limited to neurons, astrocytes, and oligodendrocytes. The proliferation rate of the cells increased by at least about 3 fold and the cells did not loss their capacity to differentiate. Preferably, the proliferation rate of the cells when cultured according to the methods of the present invention is enhanced at least about 7 fold, more preferably at least about 10 folds even more preferably at least about 15 folds most preferably at least about 30 fold where the cells do not loss their capacity to differentiate.

In yet another aspect of the present invention, the cells expand about 15-17 fold when cultured as an adherent cell population on a coated surface in the presence of LIF.

In another aspect of the invention, the cells expand about 5-7 fold when cultured as an adherent cell population on a coated surface in the absence of LIF.

In a yet another aspect of the invention, the cells expand slightly more than about 5-7 fold (i.e. about 6-8 fold) when cultured as an adherent cell population on a uncoated surface in the presence of LIF.

In a further aspect of the invention, the cells expand about 3-5 fold when cultured as an adherent cell population on an uncoated surface in the absence of LIF.

In another aspect of the present invention, the doubling time for NSCs can be modulated using methods disclosed herein. For example, NSCs cultured as an adherent cell population on a coated surface in the presence of LIF were observed to have a doubling time of about 20-24 hours. The doubling time of NSCs cultured in the absence of LIF was observed to be about 60 hours. The doubling time of NSCs cultured according to the LIF+/− regimen (grown in the presence of LIF for a period of time and then subsequently grown in the absence of LIF for a period of time) was observed to be about 28-36 hours. In one aspect, the doubling time for NSCs cultured according to the LIF+/− regimen is about 30-36 hours. In another aspect, the doubling time for NSCs cultured according to the LIF+/− regimen is about 28-30 hours.

The present invention further provides a novel method and growth medium for inducing proliferation of NSCs at an increased proliferation rate (a decreased doubling time) that can provide a larger number of NSCs compared to the number of cells generated using methods known in the art. The growth medium of the invention for proliferation of NSCs is a defined medium supplemented with LIF. The medium of the present invention can be used to culture any NSCs, for example short term and long term proliferation of NSCs, and the NSCs can be derived from any source including but not limited to mouse, rat, and human. In addition, NSCs and their differentiated progeny may be immortalized or conditionally immortalized using techniques known in the art. Alternatively, the NSCs can be used as primary cultures, whereby the cells have not been cultured in a manner that would transform or immortalize the NSCs.

Culture Medium

The medium useful for culturing NSCs contains LIF and markedly and unexpectedly increases the rate of proliferation of NSCs, particularly when used to culture an adherent NSC population.

When a comparison of growth rates of NSCs cultured in the presence and absence of LIF was conducted, unexpectedly it was observed that the presence of LIF in the culture medium dramatically increased the rate of cellular proliferation of the cultured NSCs, particularly the adherent NSC culture. Similarly, it was observed that the presence of LIF in the culture medium dramatically decreased the doubling time of the cultured NSCs.

The medium according to this invention comprises effective amounts of the following components useful for inducing the NSCs to proliferate:
(a) a standard culture medium that is serum-free (containing 0-0.49% serum) or serum-depleted (containing 0.5-5.0% serum), known as a basal medium, such as Iscove's modified Dulbecco's medium (“IMDM”), RPMI, DMEM, DMEMIF12, Fischer's, alpha medium, Leibovitz's, L-15, NCTC, F-10, F-12, MEM and McCoy's;
(b) a suitable carbohydrate source, such as glucose;
(c) a buffer such as MOPS, HEPES or Tris, preferably HEPES;
(d) one or more growth factors that stimulate proliferation of neural stem cells, such as EGF, bFGF, platelet derived growth factor (PDGF), nerve growth factor (NGF), and analogs, derivatives and/or combinations thereof, preferably EGF and bFGF in combination; and
(e) LIF.

Standard culture media typically contains a variety of essential components required for cell viability, including inorganic salts, carbohydrates, hormones, essential amino acids, vitamins, and the like. Preferably, DMEM or F-12 is the standard culture medium, most preferably a 50/50 mixture of DMEM and F-12. Both media are commercially available (DMEM; GIBCO, Grand Island, N.Y.; F-12, GIBCO, Grand Island, N.Y.). A premixed formulation of DMEM/F-12 is also available commercially. It is advantageous to provide additional glutamine to the medium. It is also advantageous to provide heparin in the medium. It is further advantageous to add sodium bicarbonate to the medium. It is also advantageous to add N2 supplement. Preferably, the conditions for culturing the NSCs should be as close to physiological conditions as possible. The pH of the culture medium is typically between 6-8, preferably about 7, most preferably about 7.4. Cells are typically cultured at a temperature between 30-40° C., preferably between 32-38° C., most preferably between 35-37° C. Cells are preferably grown in the presence of about 5% CO₂.

It is preferred that the concentration of LIF present in the medium of the present invention is about at least 2 ng/ml to about 20 ng/ml, preferably is about at least about 4 ng/ml to about 18 ng/ml more preferably about at least 6 ng/ml to about 16 ng/ml, even more preferably about at least 8 ng/ml to about 18 ng/ml, most preferably at least about 10 ng/ml to about 16 ng/ml. In one aspect of the present invention, the concentration of LIF is about 10 ng/ml.

The NSCs can be cultured in a growth medium supplemented with LIF for a period of time sufficient to induce enhanced proliferation of NSCs while preserving their multipotential capacities. Preferably, the NSCs are subjected to a treatment regimen comprising culturing the cells as an adherent culture in the presence of LIF for a period of time or until the cells reach a certain level of confluence before passing the cells to another coated surface. Preferably the level of confluence is greater than 70%. More preferably the level of confluence is greater than 90%. The period of time in which the cells are cultured in the medium of the present invention can be any time suitable for the culturing of cells in vitro. Based on the present disclosure, one skilled in the art would appreciate that the NSCs can be cultured in growth medium supplemented with LIF for more than 7 days. For example the NSCs can be cultured for about one week, two weeks, one month, two months, six months, or even one year (passing the cells when they become confluent); and the growth medium can be changed at anytime during the culture period.

The NSCs can be cultured in the presence of LIF continuously during the entire culture period. Alternatively, LIF can be removed from the medium at any time and the NSC can be cultured in the absence of LIF for a period of time. After a period of time of culturing the cells in the absence of LIF, LIF can again be added to the medium. This method of culturing NSCs is referred to herein as a LIF+/− regimen.

The LIF+/− regimen includes culturing NSCs in the presence of LIF for a period of time and then culturing the cell in the absence of LIF for another period of time. The period of time in which the cells are cultured in the presence of LIF can be any time suitable for the culturing of cells in vitro. Preferably the cells are cultured in the presence of LIF for about 7 days. Following the culturing of the NSCs in the presence of LIF, the cells are cultured in the absence of LIF for a period of time. Again, the period of time in which the cells are cultured in the absence of LIF can be any time suitable for the culturing of cells in vitro. Preferably the NSCs can be cultured in the absence of LIF for about 7 days. The LIF+/− regimen can be repeated once, twice, three times, or as many times necessary to generated a desirable cell population. The NSCs can be cultured according to the LIF+/− regimen for about two weeks, one month, two months, six months, or even one year; and the growth medium can be changed at anytime during the treatment regimen duration.

Following the culturing of the NSCs according to the methods disclosed herein, the NSCs can be harvested for experimental/therapeutic use immediately or they can be cryopreserved and be used at a later time.

MHC Modulation

NSCs from any source, i.e., those that are freshly isolated or cryopreserved, can be used for the methods of the present invention in order to induce enhanced proliferation of the NSCs while preserving their multipotential capacities.

In an embodiment of the present invention, MHC class II molecule expression in NSCs is modulated by culturing the NSCs according to the methods disclosed herein. Based on the disclosure herein, it was observed that the expression of MHC class II molecules by NSCs was closely regulated by LIF. When culturing NSCs in the presence of LIF, MHC class II molecules were observed to be present on the NSCs. It was also observed that the expression of MHC class II molecules by NSCs was reduced when the cells were cultured according to a the LIF+/− regimen, when compared the MHC class II molecule expression by an otherwise identical population of NSCs cultured in the presence of LIF.

As such, the present invention includes a method of culturing NSCs according to the LIF+/− regimen to induce enhanced proliferation (decrease doubling time) of the NSCs while preserving their multipotential capacities and modulating expression of MHC class molecules. A cell population resulting from the culturing of NSCs according to the methods disclosed herein is also included in the present invention. For example, the present invention includes a cell population comprising NSCs which have been cultured according to the LIF+/− regimen.

Without wishing to be bound by any particular theory, the cell population generated from using the methods herein is useful for experimental/therapeutic use because a larger number of cells can be obtain in the same amount of time when compared with the number of cells generated using methods known in the art. In addition, the cell population of the present invention is useful because the expression of MHC class molecules by the NSCs can be modulated using the methods of the invention.

Characterization

At any time point during the culturing of the cells in the presence of LIF (or during the absence of LIF or during the LIF+/− regimen), the cells can be harvested and collected for immediate experimental/therapeutic use or cryopreserved for use at a later time. In one aspect of the invention the cells are cryopreserved at any step during the culturing of the NSCs. Cryopreservation is a procedure common in the art and as used herein encompasses all procedures currently used to cryopreserve cells for future analysis and use. In another aspect, the cells can be harvested and subjected to flow cytometry to evaluate cell surface markers to assess the change in phenotype of the cells in view of the culture conditions.

NSCs cells may be characterized using any one of numerous methods in the art and methods disclosed herein. The cells may be characterized by the identification of surface and intracellular proteins, genes, and/or other markers indicative of differentiation of the cells such that they express at least one characteristic of a neuron like cell. These methods include, but are not limited to, (a) detection of cell surface proteins by immunofluorescent assays such as flow cytometry or in situ immunostaining of cell surface proteins such as nestin, MAP2, GFAP, DAKO, O4, CD45, CD86, CD14, CD133, CD80, CD34, MHC class II molecules and MHC class I molecules; (b) detection of intracellular proteins by immunofluorescent methods such as flow cytometry or in situ immunostaining using specific antibodies; (c) detection of the expression mRNAs by methods such as polymerase chain reaction, in situ hybridization, and/or other blot analysis.

Phenotypic markers of the desired cells are well known to those of ordinary skill in the art. Additional phenotypic markers continue to be disclosed or can be identified without undue experimentation. Any of these markers can be used to confirm the differentiation stage of the NSCs. Lineage specific phenotypic characteristics can include cell surface proteins, cytoskeletal proteins, cell morphology, and secretory products.

In order to identify the cellular phenotype either during proliferation or differentiation of the NSCs, various cell surface or intracellular markers may be used. When the NSCs of the invention are proliferating, nestin antibody can be used as a marker to identify undifferentiated cells.

When differentiated, most of the NSCs lose their nestin positive immunoreactivity. In particular, antibodies specific for various neuronal or glial proteins may be employed to identify the phenotypic properties of the differentiated NSCs. Neurons may be identified using antibodies to neuron specific enolase (“NSE”), neurofilament, tau, β-tubulin, or other known neuronal markers. Astrocytes may be identified using antibodies to glial fibrillary acidic protein (“GFAP”), or other known astrocytic markers. Oligodendrocytes may be identified using antibodies to galactocerebroside, 04, myelin basic protein (“MBP”) or other known oligodendrocytic markers.

It is also possible to identify cell phenotypes by identifying compounds characteristically produced by those phenotypes. For example, it is possible to identify neurons by their ability to produce neurotransmitters such as acetylcholine, dopamine, epinephrine, norepinephrine, and the like.

Specific neuronal phenotypes can be identified according to the specific products produced by those neurons. For example, GABA-ergic neurons may be identified by the production of glutamic acid decarboxylase (“GAD”) or GABA. Dopaminergic neurons may be identified by the production of dopa decarboxylase (“DDC”), dopamine or tyrosine hydroxylase (“TH”). Cholinergic neurons may be identified by the production of choline acetyltransferase (“ChAT”). Hippocampal neurons may be identified by staining with NeuN. Based on the present disclosure, one skilled in the art would appreciate that any suitable known marker for identifying specific neuronal phenotypes may be used.

The present invention also includes a cell cultured according to the methods provided herein. In one aspect of the invention, NSCs exhibit at least a decreased expression of MHC class II molecules after culturing according to the LIF+/− regimen when compared with the expression level of MHC class II molecules from an otherwise identical NSC cultured continuously in the presence of LIF.

In another aspect of the invention, the number of NSCs generated from an NSC cell population cultured at least in the presence of LIF for a period of time is greater than the number of NSCs generated from an otherwise identical NSC cell population cultured in the absence of LIF.

In yet another aspect of the invention, the number of NSCs generated from an NSC cell population cultured according to the LIF+/− regimen is greater than the number of NSCs generated from an otherwise identical NSC cell population cultured in the absence of LIF.

Methods of Using ADAS Cells

NSCs described herein may be cryopreserved according to routine procedures. Preferably, about one to ten million cells are cryopreserved in NSC medium with 10% DMSO in vapor phase of Liquid N₂. Frozen cells can be thawed by swirling in a 37° C. bath, resuspended in fresh proliferation medium, and grown as usual.

In another embodiment, this invention provides a differentiated cell culture containing previously unobtainable large numbers of neurons, as well as astrocytes and oligodendrocytes. Typically, using methods in the art, human NSC cultures form very few neurons. According to the methods disclosed herein, a larger number of neurons can be obtained because a larger number of NSCs can be generated. Thus, the methods of the present invention are highly advantageous as they facilitate the generation of a larger amount of a neuronal population prior to implantation into a patient having a disorder or disease where neuronal function has been impaired or lost.

The present invention also relates to the discovery that the expression of MHC molecules by NSCs can be modulated by culturing NSCs as an adherent cell population in the presence of LIF. The disclosure presented herein demonstrates that in addition to enhancing the proliferation of NSCs while preserving their multipotential capacity, culturing NSCs according to the methods included herein also modulates the upregulation and/or induction of MHC molecule expression by NSCs compared with the expression of MHC molecules by NSCs cultured using standard methods known in the art. That is, the present invention provides a method of culturing NSCs in a manner that provides additional benefits over the standard methods used for enhancing proliferation of NSCs in culture. These benefits include, but are not limited to enhancing the proliferation of the NSCs while maintaining the multipotential capacities of the NSCs and modulating MHC molecule expression by the NSCs. Preferably, the cells are cultured according to the LIF+/− regimen to generate a population of cells suitable for therapeutic use. Without wishing to be bound by any particular theory, the NSCs generated from using the LIF+/− regimen are also suitable for transplantation into a patient because the MHC class II molecules expressed by the NSCs are at a level that reduces the risk of host rejection of the transplanted NSCs.

The discovery that MHC molecule expression can be modulated using the methods disclosed herein provides a method of generating a population of NSCs that is useful for therapeutic, diagnostic, experimental uses and the like. For example, the decreased expression of MHC molecules by NSCs using the methods disclosed herein compared with methods known in the art provides a method of decreasing the immunogenicity of the NSCs. Preferably, the decreased expression of MHC molecules by the NSCs provides a method of increasing the success for transplantation of the NSCs into a recipient.

It has been widely established that transplantation of cells between genetically disparate individuals (allogeneic) invariably is associated with risk of graft rejection. Nearly all cells express products of the major histocompatibility complex, MHC class I molecules. Further, many cell types can be induced to express MHC class II molecules when exposed to inflammatory cytokines. Rejection of allografts is mediated primarily by T cells of both the CD4 and CD8 subclasses that recognize MHC class I and II molecules. A major goal in transplantation is the permanent engraftment of the donor graft without inducing a graft rejection immune response generated by the recipient. As such, the present invention encompasses methods for reducing and/or eliminating an immune response by cells of the recipient against grafted NSCs in the recipient by culturing the NSCs prior to transplantation using methods disclosed herein, in order to reduce the expression of MHC molecules by the NSCs. Without wishing to be bound to any particular theory, a reduction in the expression of MHC molecules by NSCs using the methods disclosed herein serves to reduce the number of MHC molecules present on the cell membrane of the NSCs thereby reducing the immunogenicity of the NSCs in the recipient.

NSCs obtained by methods of the present invention can be induced to differentiate into neurons, astrocytes, oligodendrocytes and the like by selection of culture conditions known in the art to lead to differentiation of NSCs into cells of a selected type.

NSCs cultured or expanded as described in this disclosure can be used to treat a variety of disorders known in the art to be treatable using NSCs. The NSCs are useful in these treatment methods can include those that have, and those that do not have an exogenous gene inserted therein. Examples of such disorders include but are not limited to brain trauma, Huntington's disease, Alzheimer's disease, Parkinson's disease, spinal cord injury, stroke, multiple sclerosis, cancer, CNS lysosomal storage diseases and head trauma.

The NSCs of the present invention described herein, and their differentiated progeny may be immortalized or conditionally immortalized using known techniques. Alternatively, the NSCs can be used as a primary culture, whereby the cells have not been cultured in a manner that would transform or immortalize the NSCs.

The NSCs of this invention have numerous uses, including for drug screening, diagnostics, genomics and transplantation. The cells of the present invention can be induced to differentiate into the neural cell type of choice using the appropriate media described in this invention. The drug to be tested can be added prior to differentiation to test for developmental inhibition, or added post-differentiation to monitor neural cell-type specific reactions.

Genetic Modification

The present invention is also useful for obtaining NSCs that express an exogenous gene, so that the NSCs can be used, for example, for cell therapy or gene therapy. That is, the present invention allows for the production of large numbers of NSCs which express an exogenous gene. The exogenous gene can, for example, be an exogenous version of an endogenous gene (i.e., a wild type version of the same gene can be used to replace a defective allele comprising a mutation). The exogenous gene is usually, but not necessarily, covalently linked with (i.e., “fused with”) one or more additional genes. Exemplary “additional” genes include a gene used for “positive” selection to select cells that have incorporated the exogenous gene, and a gene used for “negative” selection to select cells that have incorporated the exogenous gene into the same chromosomal locus as the endogenous gene or both.

The term “genetic modification” as used herein refers to the stable or transient alteration of the genotype of an NSC by intentional introduction of exogenous DNA. DNA may be synthetic, or naturally derived, and may contain genes, portions of genes, or other useful DNA sequences. The term “genetic modification” as used herein is not meant to include naturally occurring alterations such as that which occurs through natural viral activity, natural genetic recombination, or the like.

Exogenous DNA may be introduced to an NSC using viral vectors (retrovirus, modified herpes viral, herpes-viral, adenovirus, adeno-associated virus, lentiviral, and the like) or by direct DNA transfection (lipofection, calcium phosphate transfection, DEAE-dextran, electroporation, and the like). The genetically modified cells of the present invention possess the added advantage of having the capacity to fully differentiate to produce neurons or differentiated cells in a reproducible fashion using a number of differentiation protocols.

When the purpose of genetic modification of the cell is for the production of a biologically active substance, the substance will generally be one that is useful for the treatment of a given CNS disorder. For example, it may be desired to genetically modify cells so that they secrete a certain growth factor product.

The cells of the present invention can be genetically modified by having exogenous genetic material introduced into the cells, to produce a molecule such as a trophic factor, a growth factor, a cytokine, a neurotrophin, and the like, which is beneficial to culturing the cells. In addition, by having the cells genetically modified to produce such a molecule, the cell can provide an additional therapeutic effect to the patient when transplanted into a patient in need thereof.

As used herein, the term “growth factor product” refers to a protein, peptide, mitogen, or other molecule having a growth, proliferative, differentiative, or trophic effect on a cell. Growth factor products useful in the treatment of CNS disorders include, but are not limited to, nerve growth factor (NGF), brain-derived neurotrophic factor (BDNF), the neurotrophins (NT-3, NT-4/NT-5), ciliary neurotrophic factor (CNTF), amphiregulin, FGF-1, FGF-2, EGF, TGFα, TGFβs, PDGF, IGFs, and the interleukins; IL-2, IL-12, IL-13.

Cells can also be modified to express a certain growth factor receptor (r) including, but not limited to, p75 low affinity NGFr, CNTFr, the trk family of neurotrophin receptors (trk, trkb, trkC), EGFr, FGFr, and amphiregulin receptors. Cells can be engineered to produce various neurotransmitters or their receptors such as serotonin, L-dopa, dopamine, norepinephrine, epinephrine, tachykinin, substance-P, endorphin, enkephalin, histamine, N-methyl D-aspartate, glycine, glutamate, GABA, ACh, and the like. Useful neurotransmitter-synthesizing genes include TH, dopa-decarboxylase (DDC), DBH, PNMT, GAD, tryptophan hydroxylase, ChAT, and histidine decarboxylase. Genes that encode various neuropeptides which may prove useful in the treatment of CNS disorders, include substance-P, neuropeptide-Y, enkephalin, vasopressin, VIP, glucagon, bombesin, cholecystokinin (CCK), somatostatin, calcitonin gene-related peptide, and the like.

According to the present invention, gene constructs which comprise nucleotide sequences that encode heterologous proteins are introduced into the NSCs. That is, the cells are genetically altered to introduce a gene whose expression has therapeutic effect in the individual. According to some aspects of the invention, NSCs from the individual to be treated or from another individual, or from a non-human animal, may be genetically altered to replace a defective gene and/or to introduce a gene whose expression has therapeutic effect in the individual being treated.

In all cases in which a gene construct is transfected into a cell, the heterologous gene is operably linked to regulatory sequences required to achieve expression of the gene in the cell. Such regulatory sequences typically include a promoter and a polyadenylation signal.

The gene construct is preferably provided as an expression vector that includes the coding sequence for a heterologous protein operably linked to essential regulatory sequences such that when the vector is transfected into the cell, the coding sequence will be expressed by the cell. The coding sequence is operably linked to the regulatory elements necessary for expression of that sequence in the cells. The nucleotide sequence that encodes the protein may be cDNA, genomic DNA, synthesized DNA or a hybrid thereof or an RNA molecule such as mRNA.

The gene construct includes the nucleotide sequence encoding the beneficial protein operably linked to the regulatory elements and may remain present in the cell as a functioning cytoplasmic molecule, a functioning episomal molecule or it may integrate into the cell's chromosomal DNA. Exogenous genetic material may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be introduced into the cell.

The regulatory elements for gene expression include: a promoter, an initiation codon, a stop codon, and a polyadenylation signal. It is preferred that these elements be operable in the cells of the present invention. Moreover, it is preferred that these elements be operably linked to the nucleotide sequence that encodes the protein such that the nucleotide sequence can be expressed in the cells and thus the protein can be produced. Initiation codons and stop codons are generally considered to be part of a nucleotide sequence that encodes the protein. However, it is preferred that these elements are functional in the cells. Similarly, promoters and polyadenylation signals used must be functional within the cells of the present invention. Examples of promoters useful to practice the present invention include but are not limited to promoters that are active in many cells such as the cytomegalovirus promoter, SV40 promoters and retroviral promoters. Other examples of promoters useful to practice the present invention include but are not limited to tissue-specific promoters, i.e. promoters that function in some tissues but not in others; also, promoters of genes normally expressed in the cells with or without specific or general enhancer sequences. In some embodiments, promoters are used which constitutively express genes in the cells with or without enhancer sequences. Enhancer sequences are provided in such embodiments when appropriate or desirable.

The cells of the present invention can be transfected using well known techniques readily available to those having ordinary skill in the art. Exogenous genes may be introduced into the cells using standard methods where the cell expresses the protein encoded by the gene. In some embodiments, cells are transfected by calcium phosphate precipitation transfection, DEAE dextran transfection, electroporation, microinjection, liposome-mediated transfer, chemical-mediated transfer, ligand mediated transfer or recombinant viral vector transfer.

In some embodiments, recombinant adenovirus vectors are used to introduce DNA with desired sequences into the cell. In some embodiments, recombinant retrovirus vectors are used to introduce DNA with desired sequences into the cells. In some embodiments, standard CaPO₄, DEAE dextran or lipid carrier mediated transfection techniques are employed to incorporate desired DNA into dividing cells. Standard antibiotic resistance selection techniques can be used to identify and select transfected cells. In some embodiments, DNA is introduced directly into cells by microinjection. Similarly, well-known electroporation or particle bombardment techniques can be used to introduce foreign DNA into the cells. A second gene is usually co-transfected or linked to the therapeutic gene. The second gene is frequently a selectable antibiotic-resistance gene. Transfected cells can be selected by growing the cells in an antibiotic that will kill cells that do not take up the selectable gene. In most cases where the two genes are unlinked and co-transfected, the cells that survive the antibiotic treatment have both genes in them and express both of them.

Use of Isolated Neural Stem Cells

Isolated neural stem cells are useful in a variety of ways. These cells can be used to reconstitute cells in a mammal whose cells have been lost through disease or injury. Genetic diseases may be treated by genetic modification of autologous or allogeneic neural stem cells to correct a genetic defect or to protect against disease. Diseases related to the lack of a particular secreted product such as a hormone, an enzyme, a growth factor, or the like may also be treated using NSCs. CNS disorders encompass numerous afflictions such as neurodegenerative diseases (i.e. Alzheimer's and Parkinson's), acute brain injury (i.e. stroke, head injury, cerebral palsy) and a large number of CNS dysfunctions (i.e. depression, epilepsy, and schizophrenia). Diseases including but are not limited to Alzheimer's disease, multiple sclerosis (MS), Huntington's Chorea, amyotrophic lateral sclerosis (ALS), and Parkinson's disease, have all been linked to the degeneration of neural cells in particular locations of the CNS, leading to the inability of these cells or the brain region to carry out their intended function. NSCs isolated and cultured as described herein can be used as a source of progenitor cells and committed cells to treat these diseases.

The NSCs cultured as described herein may be frozen at liquid nitrogen temperatures and stored for long periods of time, after which they can be thawed and are capable of being reused. The cells are usually stored in 10% DMSO and 90% complete growth medium. Once thawed, the cells may be expanded using the methods described elsewhere herein.

It is envisioned that NSCs obtained using the methods of the present invention can be induced to differentiate into neurons, astrocytes, oligodendrocytes and the like by selection of culture conditions known in the art to lead to differentiation of NSCs into cells of a selected type. For example, NSCs can be induced to differentiate by plating the cells on a coated surface, preferably polyomithine or poly-L-lysine (PPL), in the absence of growth factors but in the presence of 10% fetal bovine serum (FBS). Differentiation can also be induced by plating the cells on a fixed substrate such as flasks, plates, or coverslips coated with an ionically charged surface such as poly-L-lysine and poly-L-ornithine and the like. Other substrates may be used to induce differentiation such as collagen, fibronectin, laminin, MATRIGEL™ (Collaborative Research), and the like.

A preferred method for inducing differentiation of the neural stem cell progeny comprises culturing the cells on a fixed substrate in a culture medium that is free of proliferation-inducing growth factor. After removal of the proliferation-inducing growth factor, the cells adhere to the substrate (i.e. poly-ornithine-treated plastic or glass), flatten, and begin to differentiate into neurons and glial cells. At this stage, the culture medium may contain serum such as 0.5-1.0% fetal bovine serum (FBS). However, for certain uses, if defined conditions are required, serum should not be used. Within 2-3 days, most or all of the neural stem cell progeny begin to lose immunoreactivity for nestin and begin to express antigens specific for neurons, astrocytes or oligodendrocytes as determined by immunocytochemistry techniques well known in the art. In particular, cellular markers for neurons include but not limited to neuron-specific enolase (NSE), neurofilament (NF), β-tubulin, MAP-2; and for glial, GFAP, galactocerebroside (GalC) (a myelin glycolipid identifier of oligodendrocytes), and the like.

NSCs cultured or expanded as described in this disclosure can be used, as cultured, or they can be used following differentiation into selected cell types, to treat a variety of disorders known in the art to be treatable using NSCs. The NSCs that are useful in these treatment methods include those that have, and those that do not have an exogenous gene inserted therein. Examples of disorders that can be treated include but are not limited to brain trauma, Huntington's Chorea, Alzheimer's disease, Parkinson's disease, spinal cord injury, stroke, multiple sclerosis, head trauma and other such diseases and/or injuries where the replacement of tissue by the cells of the present invention can result in a treatment or alleviation of the disease and/or injuries.

The present invention encompasses methods for administering the cells of the present invention to an animal, including humans, in order to treat diseases where the introduction of new, undamaged cells will provide some form of therapeutic relief.

The cells of the present invention can be administered as an NSC or an NSC that has been induced to differentiate to exhibit at least one characteristic of a neuronal like cell. The skilled artisan will readily understand that NSCs can be administered to an animal as a differentiated cell, for example, a neuron, and will be useful in replacing diseased or damaged neurons in the animal. Additionally, an NSC can be administered and upon receiving signals and cues from the surrounding milieu, can differentiate into a desired cell type dictated by the neighboring cellular milieu.

The cells can be prepared for grafting to ensure long term survival in the in vivo environment. For example, cells are propagated in a suitable culture medium for growth and maintenance of the cells and are allowed to grow to confluency. The cells are loosened from the culture substrate using, for example, a buffered solution such as phosphate buffered saline (PBS) containing 0.05% trypsin supplemented with 1 mg/ml of glucose; 0.1 mg/ml of MgCl₂, 0.1 mg/ml CaCl₂ (complete PBS) plus 5% serum to inactivate trypsin. The cells can be washed with PBS and are then resuspended in the complete PBS without trypsin and at a selected density for injection.

In addition to PBS, any osmotically balanced solution which is physiologically compatible with the host subject may be used to suspend and inject the donor cells into the host. Formulations of a pharmaceutical composition suitable for parenteral administration comprise the active ingredient, i.e. the cells, combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The invention also encompasses grafting NSCs (or differentiated NSCs) in combination with other therapeutic procedures to treat disease or trauma to the CNS and peripheral regions. Thus, the cells of the invention may be co-grafted with other cells, both genetically modified and non-genetically modified cells which exert beneficial effects on the patient. Therefore the methods disclosed wherein can be combined with other therapeutic procedures as would be understood by one skilled in the art once armed with the teachings provided herein.

The cells can be transplanted as a mixture/solution comprising of single cells or a solution comprising a suspension of a cell aggregate. Such aggregate can be approximately 10-500 micrometers in diameter, and, more preferably, about 40-50 micrometers in diameter. A cell aggregate can comprise about 5-100, more preferably, about 5-20, cells per sphere. The density of transplanted cells can range from about 10,000 to 1,000,000 cells per microliter, more preferably, from about 25,000 to 500,000 cells per microliter.

Transplantation of the cells of the present invention can be accomplished using techniques well known in the art as well as those described herein or as developed in the future. The present invention comprises a method for transplanting, grafting, infusing, or otherwise introducing NSCs or differentiated NSCs into an animal, preferably, a human. Exemplified below are methods for transplanting the cells into the brains of both rodents and humans, but the present invention is not limited to such anatomical sites or to those animals. Also, methods for bone transplants are well known in the art and are described in, for example, U.S. Pat. No. 4,678,470, pancreas cell transplants are described in U.S. Pat. No. 6,342,479, and U.S. Pat. No. 5,571,083, teaches methods for transplanting cells, such as NSCs, to any anatomical location in the body.

The cells may also be encapsulated and used to deliver biologically active molecules, according to known encapsulation technologies, including microencapsulation (see, i.e., U.S. Pat. Nos. 4,352,883; 4,353,888; and 5,084,350, herein incorporated by reference), or macroencapsulation (see, i.e., U.S. Pat. Nos. 5,284,761; 5,158,881; 4,976,859; and 4,968,733; and International Publication Nos. WO 92/19195; WO 95/05452, all of which are incorporated herein by reference). For macroencapsulation, cell number in the devices can be varied; preferably, each device contains between 10³-10⁹ cells, most preferably, about 105 to 10⁷ cells. Several macroencapsulation devices may be implanted in the patient. Methods for the macroencapsulation and implantation of cells are well known in the art and are described in, for example, U.S. Pat. No. 6,498,018.

The cells of the present invention can also be used to express a foreign protein or molecule for a therapeutic purpose or for a method of tracking their integration and differentiation in a patient's tissue. Thus, the invention encompasses expression vectors and methods for the introduction of exogenous DNA into the cells with concomitant expression of the exogenous DNA in the cells such as those described, for example, in Sambrook et al. (2002, Molecular Cloning: A Laboratory Manual, Cold Spring Harbor Laboratory, New York), and in Ausubel et al. (1997, Current Protocols in Molecular Biology, John Wiley & Sons, New York).

The isolated nucleic acid can encode a molecule used to track the migration, integration, and survival of NSCs once they are placed in the patient, or they can be used to express a protein that is mutated, deficient, or otherwise dysfunctional in the patient. Proteins for tracking can include, but are not limited to green fluorescent protein (GFP), any of the other fluorescent proteins (i.e., enhanced green, cyan, yellow, blue and red fluorescent proteins; Clontech, Palo Alto, Calif.), or other tag proteins (i.e., LacZ, FLAG-tag, Myc, His₆, and the like) disclosed elsewhere herein. Alternatively, the isolated nucleic acid introduced into the NSC cell can include, but are not limited to CFTR, hexosaminidase, and other gene-therapy strategies well known in the art or to be developed in the future.

Tracking the migration, differentiation and integration of the cells of the present invention is not limited to using detectable molecules expressed from a vector or virus. The migration, integration, and differentiation of a cell can be determined using a series of probes that would allow localization of transplanted NSCs. Such probes include those for human-specific Alu, which is an abundant transposable element present in about 1 in every 5000 base pairs, thus enabling the skilled artisan to track the progress of an NSC transplant. Tracking an NSC transplant may further be accomplished by using antibodies or nucleic acid probes for cell-specific markers detailed elsewhere herein, such as, but not limited to, NeuN, MAP2, neurofilament proteins, and the like.

Expression of an isolated nucleic acid, either alone or fused to a detectable tag polypeptide, in NSCs can be accomplished by generating a plasmid, viral, or other type of vector comprising the desired nucleic acid operably linked to a promoter/regulatory sequence which serves to drive expression of the protein, with or without tag, in NSCs in which the vector is introduced. Many promoter/regulatory sequences useful for driving constitutive expression of a gene are available in the art and include, but are not limited to, for example, the cytomegalovirus immediate early promoter enhancer sequence, the SV40 early promoter, as well as the Rous sarcoma virus promoter, and the like. Moreover, inducible and tissue specific expression of the desired nucleic acid may be accomplished by placing the desired nucleic acid, with or without a tag, under the control of an inducible or tissue specific promoter/regulatory sequence. Examples of tissue specific or inducible promoter/regulatory sequences which are useful for this purpose include, but are not limited to the MMTV LTR inducible promoter, and the SV40 late enhancer/promoter. In addition, promoters which are well known in the art that are induced in response to inducing agents such as metals, glucocorticoids, hormones, antibiotics (such as tetracycline) and the like, are also contemplated in the invention. Thus, it will be appreciated that the invention includes the use of any promoter/regulatory sequence, which is either known or unknown, and which is capable of driving expression of the desired protein operably linked thereto.

Where the expression of a dysfunctional protein causes a disease, disorder, or condition associated with such expression, the expression of the corrected protein from NSCs driven by a promoter/regulatory sequence can provide useful therapeutics including, but not limited to, gene therapy. Diseases, disorders and conditions associated with a dysfunctional protein are disclosed elsewhere herein and are well known in the art.

Selection of any particular plasmid vector or other DNA vector is not a limiting factor in this invention and a vast plethora of vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (2002, Molecular Cloning: A Laboratory Manual, Cold Spring Harbor Laboratory, New York), and in Ausubel et al. (1997, Current Protocols in Molecular Biology, John Wiley & Sons, New York).

The invention thus includes an NSC comprising a vector encoding an isolated nucleic acid encoding a desired protein or other molecule. The incorporation of a desired nucleic acid into a vector and the choice of vectors is well-known in the art as described in, for example, Sambrook et al. (2002, Molecular Cloning: A Laboratory Manual, Cold Spring Harbor Laboratory, New York), and in Ausubel et al. (1997, Current Protocols in Molecular Biology, John Wiley & Sons, New York).

The nucleic acids encoding the desired protein may be cloned into various plasmid vectors. However, the present invention should not be construed to be limited to plasmids, or to any particular vector. Instead, the present invention encompasses a wide plethora of vectors which are readily available and/or well-known in the art or such as are developed in the future.

The invention also includes a recombinant NSC comprising, inter alia, an isolated nucleic acid. In one aspect, the recombinant cell can be transiently transfected with a plasmid encoding a portion of a desired nucleic acid. The nucleic acid need not be integrated into the cell genome nor does it need to be expressed in the cell.

The invention includes an NSC which, when a transgene of the invention is introduced therein, and the protein encoded by the desired gene is expressed therefrom, where it was not previously present or expressed in the cell or where it is now expressed at a level or under circumstances different than that before the transgene was introduced, a benefit is obtained. Such a benefit may include the fact that there has been provided a system wherein the expression of the desired gene can be studied in vitro in the laboratory or in a mammal in which the cell resides, a system wherein cells comprising the introduced gene can be used as research, diagnostic and therapeutic tools, and a system wherein mammal models are generated which are useful for the development of new diagnostic and therapeutic tools for selected disease states in a mammal.

An NSC expressing a desired isolated nucleic acid can be used to provide the product of the isolated nucleic acid to a cell, tissue, or whole mammal where a higher level of the gene product can be useful to treat or alleviate a disease, disorder or condition associated with abnormal expression, and/or activity. Therefore, the invention includes an NSC expressing a desired isolated nucleic acid where increasing expression, protein level, and/or activity of the desired protein can be useful to treat or alleviate a disease, disorder or condition.

The following examples are presented in order to more fully illustrate the preferred embodiments of the invention. These examples should in no way be construed as limiting the scope of the invention, as defined by the appended claims.

### EXAMPLES

### Example 1

### Effect of LIF and Coating Flasks on hNSC Growth

In the present Example, human NSCs were grown as an adherent population on coated dishes. The combination of growing human fetal NSCs as an adherent population on a coated dish in the presence of LIF enhanced the proliferation rate of the cells by about 3-7 fold and the cells did not lose their capacity to differentiate into neurons, astrocytes, oligodendrocyte and the like.

The materials and methods used in the experiments presented in this Example are now described.

Isolation and Culturing of Human Fetal Neural Stem Cells

Human fetal brain tissue was purchased from Advanced Bioscience Resources (Alameda, Calif.). The tissue was washed with phosphate buffered saline (PBS) supplemented with penicillin/streptomycin solution. The tissue was then placed in a sterile Petri dish in cold PBS supplemented with penicillin/streptomycin to further clean the tissue and remove the menninges. The tissue was teased with a pair of forceps to break the tissue into smaller pieces. The tissue can further be dissociated using a Pasteur pipette (about 20 times) to triturate the tissue. The tissue can again be further dissociated using a Pasteur pipette fire-polished to significantly reduce the bore size (20 times) to triturate the tissue.

The resulting cells were pelleted by centrifugation at 1000 r.p.m. for 5 minutes at room temperature. The cell pellet was resuspended in 10 ml of growth medium (DMEM/F 12 (Invitrogen), 8 mM glucose, glutamine, 20 mM sodium bicarbonate, 15 mM HEPES, 8 μg/ml Heparin (Sigma), N2 supplement (Invitrogen), 10 ng/ml bFGF (Peprotech), 20 ng/ml EGF (Peprotech)). The cells were plated on a coated T-25 cm² flask with vented cap and grown in a 5% CO₂ incubator at 37° C. Cells grown in the presence of LIF (Sigma) were plated in complete growth medium with 10 ng/ml LIF after growing them initially (preferably after 1-2 passages) in the presence of bFGF and EGF alone. Cultures were fed every other day by replacing 50% of the medium with fresh complete growth medium.

To passage the cells, the cells were trypsinized using 0.05% trypsin-EDTA in PBS for 2-3 minutes followed by addition of soybean trypsin inhibitor to inactivate the trypsin. The cells were pelleted at 1200 r.p.m. for 5 minutes at room temperature and then were resuspended in growth medium. Cells were plated at 100,000-125,000 cells/cm² on coated flasks. Cells were cryopreserved in 10% DMSO+90% complete growth medium.

Coating of Flasks

To coat a flask, 15 μg/ml polyornithine (Sigma) in 1×PBS was added to the flask and the flask was incubated overnight at 37° C. in an incubator. Excess polyornithine was removed from the flask the next day. The flask was washed three times with 1×PBS and 10 μg/ml human fibronectin (Chemicon) in 1×PBS was added to the flask, and the flask was incubated for at least 4 hrs at 37° C. Before using the “coated” flask to culture the cells of the present invention, excess fibronectin was removed from the flask.

Differentiation Assay

NSCs were differentiated in the presence of 10 ng/ml brain-derived neurotrophic factor (BDNF) (Peprotech). The cells were plated on a coated chamber slides at 100,000 cells/chamber in complete growth medium without bFGF or EGF for 4 days. After 4 days, complete growth medium was replaced with neurobasal medium comprising 20 nM GlutaMax™ (Gibco) and B-27 supplements for another 4 days followed by neurobasal+20 nM GlutaMax™ (Gibco)+B-27+10 ng/ml BDNF for 1 week. The cells were differentiated for 15 days before the cells were fixed in preparation for immunostaining. Cells were fed 3 times a week with the appropriate medium.

Immunostaining

The cultures were fixed for 15 minutes at room temperature with 4% paraformaldehyde in 1×PBS then washed three times with 1×PBS for 10 minutes each. In preparation for immunostaining, cells were treated with 0.1% Triton X-100 to permeablize the cells; the cells were then blocked using 5% normal goat serum in 1×PBS for 2 hours at room temperature, followed by incubation with primary antibodies diluted in 5% normal goat serum in 1×PBS at 4° C. overnight. The next day, after removing the primary antibodies, cells were washed three times with 1×PBS and incubated in diluted secondary antibodies for 2 hours at room temperature. The secondary antibodies were washed with 1×PBS three times for 10 minutes each time. Stained cells were mounted with Fluormount G (Southern Biotechnology Associates) and coversliped.

The primary antibodies used were human specific nestin, 1:10 (R&D Systems); MAP2, 1:500 (Sigma); GFAP (astrocyte cytoskeletal marker), 1:1000 (DAKO); 04, 1:100 (Chemicon); BrdU-Alexa 488 conjugated 1:20 (Molecular Probes). The secondary antibodies used in these experiments were Alexa Fluor 488 chicken anti-mouse, 1:500 (Molecular Probes) and Alexa Fluor 594 chicken anti-rabbit, 1:500 (Molecular Probes).

To quantitate different cell phenotypes, cell nuclei were stained with DAPI (Sigma) for 10 minutes at room temperature. For each quantitation, three different fields from each chamber were counted using a 40× objective. Total number of cells was counted by counting the DAPI stained nuclei. Nestin-immunoreactive (ir) or GFAP-ir and MAP2-ir cells were also counted. Percentage of each phenotype was calculated.

The Results of the experiments presented in this Example are now described.

Generation of Human Neural Stem Cell Cultures

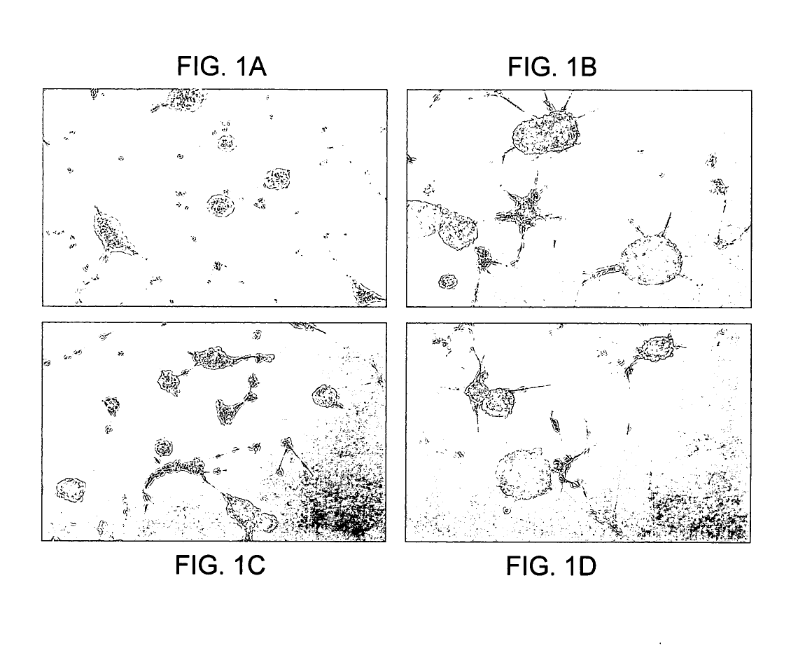

Human neural stem cell cultures were derived from human fetal brain samples and grown in the presence of bFGF and EGF. Each culture was designated by anatomical tissue identification (FB=fore brain, HB=hind brain, WB=whole brain, SC=spinal cord) and sample number (001-025). Two parallel cultures with and without hLIF were generated from some samples. All cultures were grown on coated flasks. Initially, cells attached to the flask but small clusters of cells also formed after continued proliferation and many large clusters were observed to detach from the surface of the flask and float freely. After 14-16 days in culture, the cells were passaged. FIG. 1 depicts cells of THD-hWB-015 and THD-hFB-017 cultures in the presence and absence of LIF. Some of these cultures were continuously maintained in culture for over 7 months or up to passage 17 and also were cryopreserved. Cells thawed from cryopreserved samples exhibited over 90% viability and were successfully expanded.

Effect of LIF and Coating on the Growth Rate

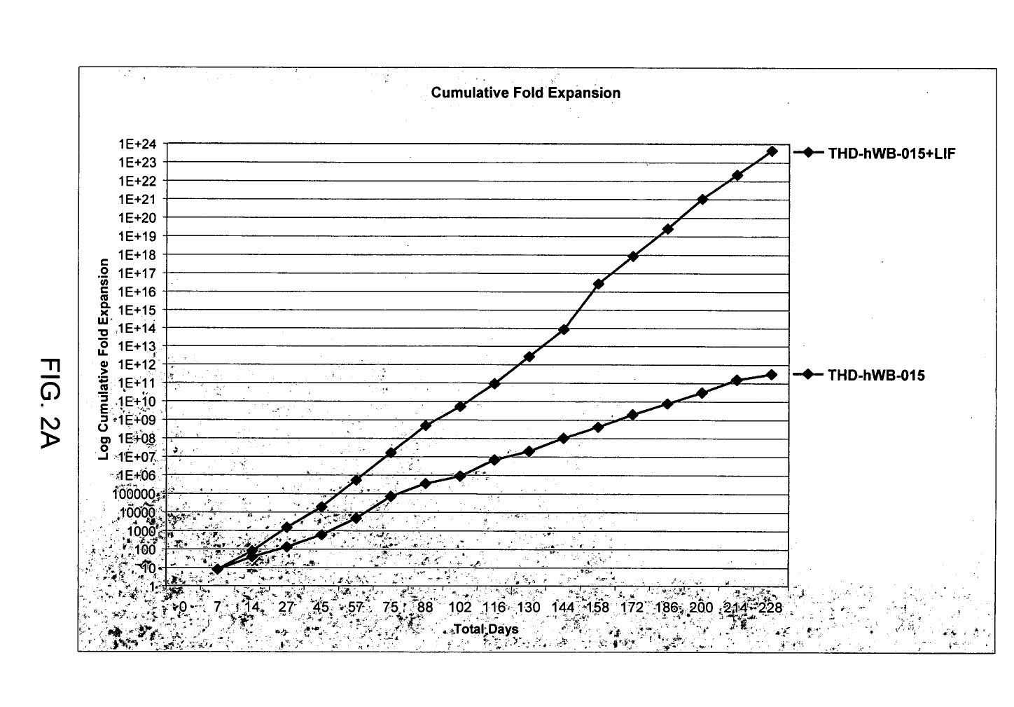

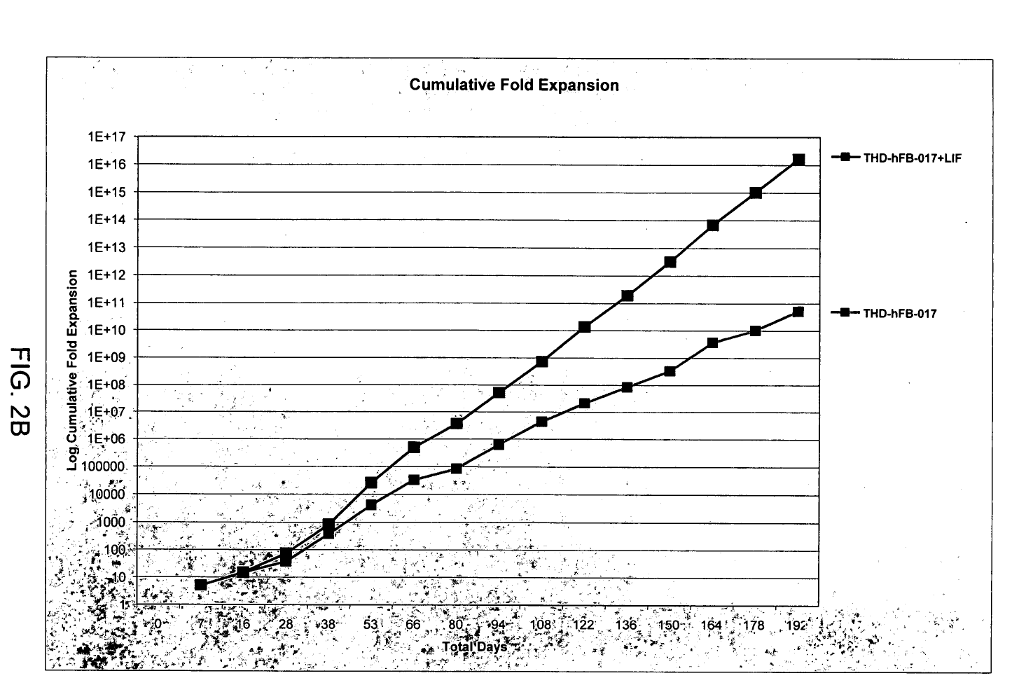

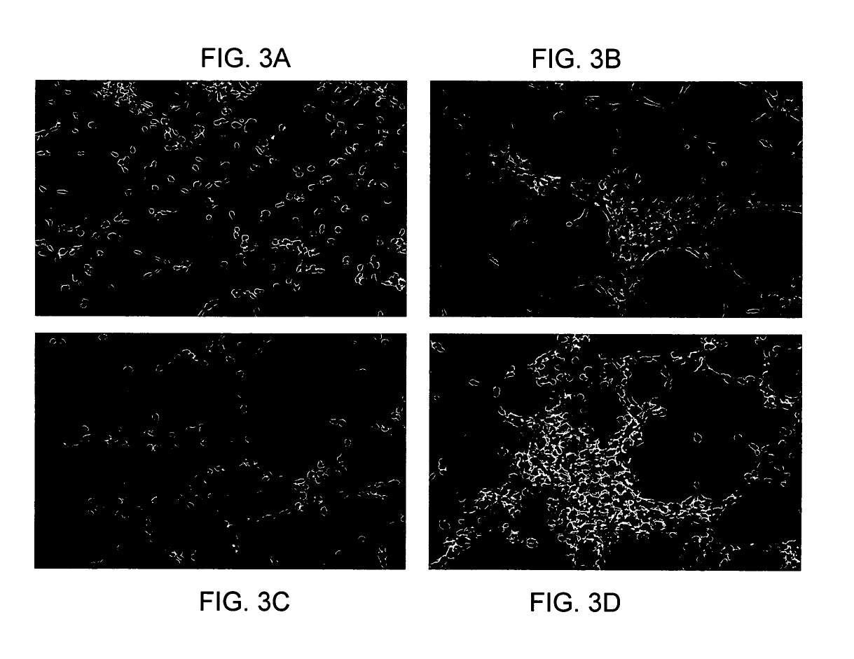

At every passage, the total number of viable cells was counted and the total fold expansion was calculated in the presence or absence of LIF. Resulting growth curves were plotted using Microsoft Excel (FIG. 2). Initially, the first 3-4 passage cultures (THD-hWB-015 and THD-hFB-017) grown in the presence of bFGF and EGF alone, demonstrated similar expansion rates when compared with cells grown in the presence of bFGF, EGF and LIF. The latter passaged cells grown in the absence of LIF demonstrated a 3-5 fold slower growth rate compared to cells grown in the presence of LIF over a period of 14-16 days. THD-hWB-015 cells exhibited a greater difference in the growth rate compared with THD-hFB-017. BrdU incorporation in these cultures was assessed to determine whether the difference in the fold expansion was due to active proliferation. There was on average 20-30% cells incorporating BrdU in the absence of LIF while 70-85% cells incorporated BrdU in the presence of LIF (FIG. 3).

Effect of LIF on Neural Stem Cell Marker Nestin Expression

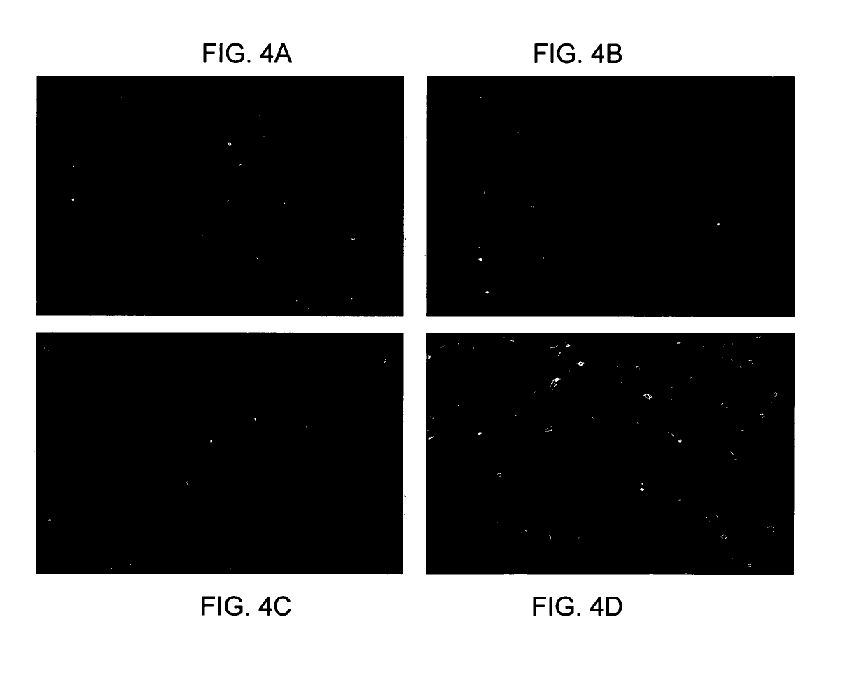

Nestin is an intermediate filament protein found in many types of undifferentiated CNS cells. At every third passage, cells were tested for nestin expression by immunocytochemical analysis. Cells were plated on coated chamber slides in complete growth medium for 24 hrs prior to fixing. Fixed cells were stained with anti-nestin and anti-GFAP antibodies while cell nuclei were stained with 4′,6′-diamidino-2-phenylindole hydrochloride (DAPI) to count total number of cells. In the case of undifferentiated THD-hWB-015 cultures in the absence of LIF but in the presence of bFGF and EGF, 86% cells were nestin alone positive, while 8-10% cell were nestin positive as well as GFAP positive, while 3-4% cells were GFAP positive alone. In the case of THD-hWB-015+LIF+bFGF+EGF, 98% cells were both GFAP and nestin positive, while 1-2% cells were GFAP positive alone (FIG. 4). GFAP, glial fibrillary acidic protein, is a marker for astrocytes.

Effect of LIF on Multipotency of NSC Cultures

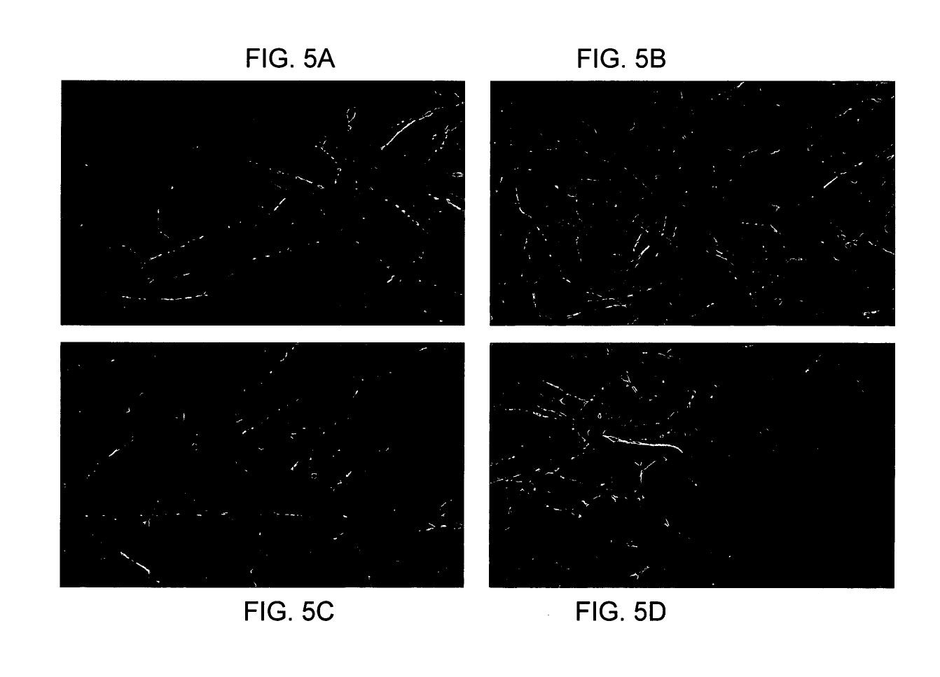

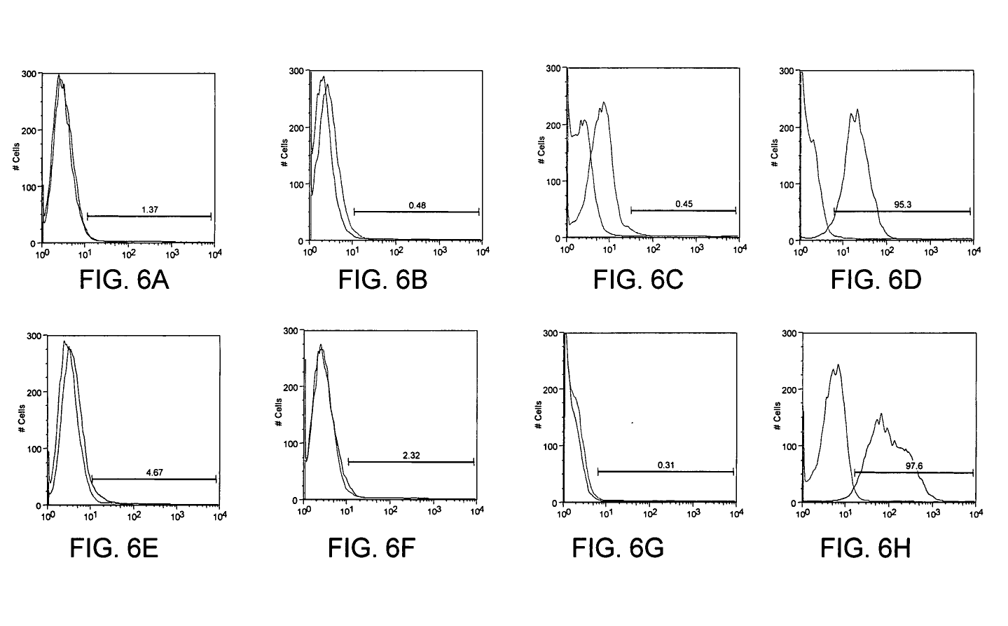

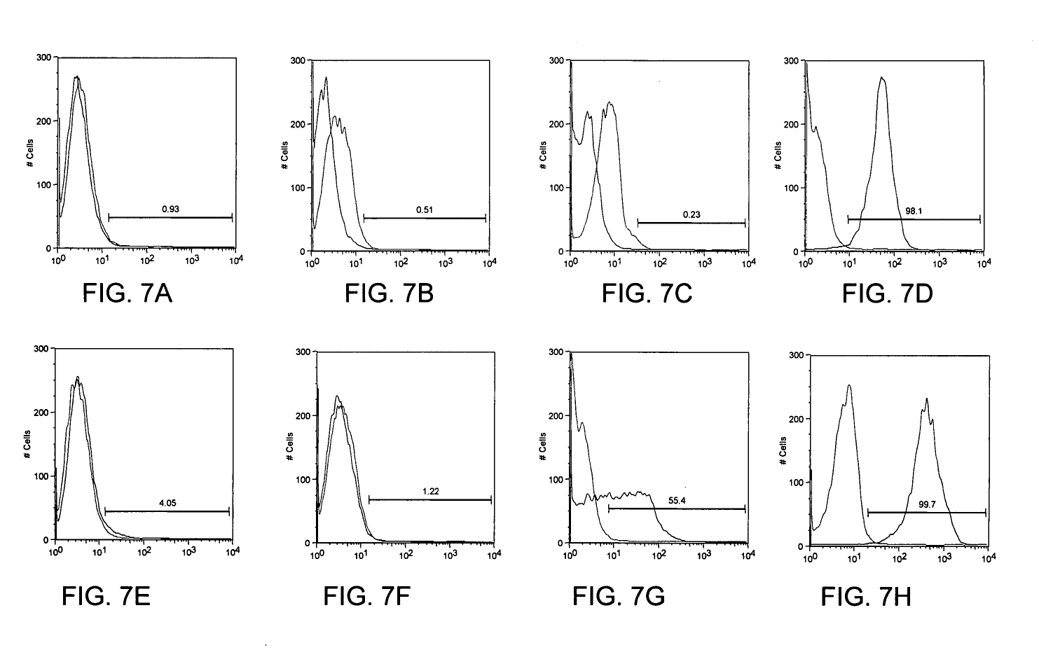

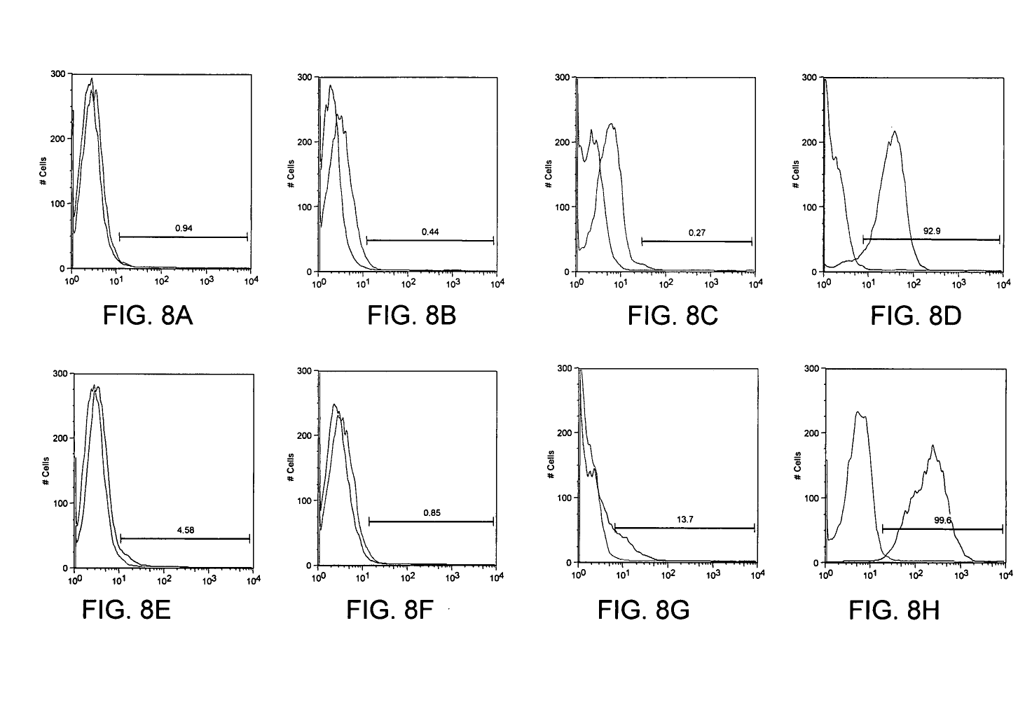

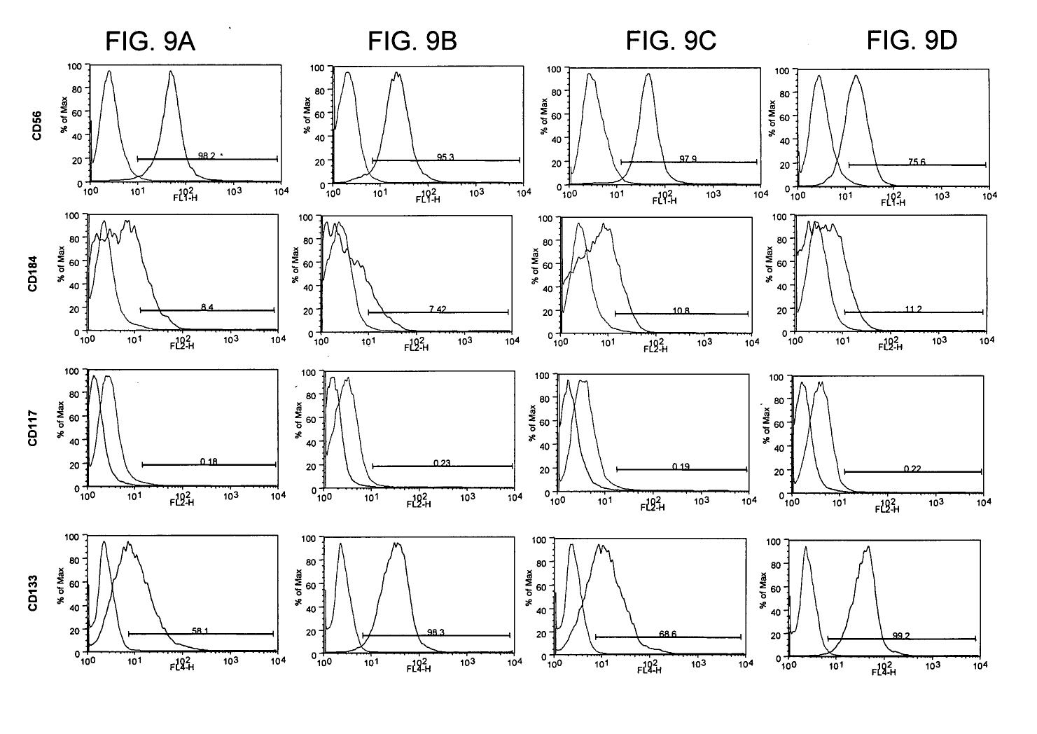

In addition to nestin expression, the differentiation potential of NSC cultures was evaluated following incubation with LIF. Cells were plated on coated chamber slides and allowed to differentiate for 14 days by withdrawing the growth factors and growing them in a differentiation medium. Cells were fixed and stained for anti-MAP2 and anti-GFAP antibodies. The total number of cells was visualized by staining the nuclei with DAPI. After differentiation of THD-hWB-015 cells, 30-40% of cells were GFAP positive, while 60-68% cells were MAP2 positive. The same culture grown in the presence of LIF (THD-hWB-015+LIF) upon differentiation exhibited 30-45% GFAP positive cells and 55-66% MAP2 positive cells. For THD-hFB-017, 60-70% cells were MAP2 positive and 25-30% were GFAP positive. In the case of THD-hFB-017+LIF, 50-60% cells were MAP2 positive and 40-45% cells were GFAP positive (FIG. 5).

To assess the long term effects of LIF on multipotency of these cultures, cells from late passage, for example as late as passage 15, from both THD-hWB-015 and THD-hFB-017 cultures, were tested. Significant differences were not observed in the total number of neurons and astrocytes in these late passage cultures when compared with earlier passages. All the cultures were also evaluated for oligodendrocyte upon differentiation. Very few 04-immunoreactive cells were observed both in the presence and absence of LIF.

### Example 2

### Effect of LIF on Growth and MHC Class II Molecule Expression

In the present Example, human NSCs were cultured using methods described elsewhere herein. The effects of LIF on the growth rate and the expression of certain genes by NSCs were determined. In particular, the effects of the continuous presence of LIF in the culture medium on the growth rate and the expression of certain genes by NSCs were assessed. Three parallel cultures were grown on coated dishes using methods discussed elsewhere herein. Cells were grown (i) in the presence of LIF (LIF+), (ii) in the absence of LIF (LIF−) or (iii) in the presence of LIF for about 7 days then grown in the absence of LIF for about 7 more days (LIF+/−). All cultures were grown for a total of 14 days. The expansion rate was calculated and the expression of particular stem cell and immunogenic markers on these cultures were assessed using methods discussed elsewhere herein.

It was observed that the doubling time for NSCs cultured in the presence of LIF was approximately 20-24 hours, 60 hours in the absence of LIF and 28-30 hours in the LIF+/−culture. In independent experiments, it was also observed that the doubling time of NSCs cultured according to the LIF+/− regimen was approximately 30-36 hours. Without wishing to be bound by any particular theory, it is believed that the doubling time relates to the passage number of the NSCs. In all the cases NSCs were CD34−, CD86−, CD80− but greater than 90% cells were CD133+. Among the genes tested, the expression of MHC Class II molecule by the NSCs was observed to be closely regulated by LIF. In the presence of LIF, MHC Class I molecule, MHC Class II molecule and CD133 molecules were displayed on the cells. There were very few MHC Class II molecules displayed in LIF− and LIF+/−cultures, but these cultures also displayed MHC Class I and CD133.

When the cells were grown in the presence of LIF for about 7 days and then grown in the absence of LIF for about another 7 day, it was observed that the cells expanded 20 fold as compared to 30 fold when grown in the presence of LIF for about 14 days. It was also observed that the expression of MHC II molecule by NSCs was reduced when the cells were grown in the presence of LIF for about 7 days and then grown in the absence of LIF for another 7 days as compared with the MHC II expression by cells grown in the presence of LIF for about 14 days.

One skilled in the art would appreciate base upon the present disclosure that the low expression of MHC II is desirable for using the cells for allogenic or autologous transplantation, as the reduced MHC II expression potentially reduces or eliminates the need for immunosupression.

### Example 3

### Characterization of Human NSCs (FACS Analysis of Human NSCs)

Cells were harvested and FACS analysis was carried out on approximately 2×10⁶ human NSCs. The cells were washed once in 2 ml flow wash buffer [1×DPBS (Hyclone, Logan, Utah), 0.5% BSA (Sigma, St. Louis, Mo.) and 0.1% sodium azide (Sigma, St. Louis, Mo.)], the cells were pelleted using centrifugation at 550×g for 5 minutes then suspended in blocking buffer [wash buffer+25 μg/ml mouse Ig (Sigma, St. Louis, Mo.)] at 1×10⁷ cells/ml. The cells were distributed in 100 μl aliquots, placed on ice and allowed to block for 10 minutes prior to monoclonal antibody addition. Propidium iodide (PI) analysis of cell viability was performed immediately following this incubation. Antibody was added to the cell suspensions at 10 μg/ml and incubated on ice for 30 minutes. The cells were washed in 2 ml wash buffer and fixed in 200 μl 1% paraformaldehyde (Electron Microscope Sciences). The NSC populations were analyzed for surface expression of the following antigens for phenotypic characterization: CD14 (Becton Dickinson (BD), Lincoln Park, N.J.), CD34 (BD), CD45 (BD), CD80 (Caltag Laboratories, Burlingame, Calif.), CD86 (Caltag), CD133 (Miltenyi Biotech Inc., Auburn, Calif.), HLA-A,B,C (BD) and HLA-DR (BD) (all antibodies were purchased from BD-Pharmingen unless otherwise stated). Final analysis of expression was based on percent (+) events as well as mean fluorescence intensity values relative to their respective isotype controls. Data was acquired on a Becton Dickinson FACSCaliber flow cytometer using Cell Quest acquisition software (BDIS) and analysis was performed using Flow Jo analysis software (Tree Star).

The disclosures of each and every patent, patent application, and publication cited herein are hereby incorporated herein by reference in their entirety.

It will be apparent to those skilled in the art that various modifications and variations can be made in the methods and compositions of the present invention without departing from the spirit or scope of the invention. Thus, it is intended that the present invention cover the modifications and variations of the present invention provided they come within the scope of the appended claims and their equivalents.

1. A composition comprising an in vitro adherent culture comprising a Neural Stem Cell (NSC), wherein said NSC proliferates in the presence of LIF while maintaining multipotentiality of said NSC.

2. The composition of claim 1, wherein said NSC adheres to a surface coated with polyornithine and fibronectin.

3. The composition of claim 1, wherein said NSC is derived from a human.

4. The composition of claim 1, wherein exogenous genetic material has been introduced into said NSC.

5. A method for the in vitro expansion and maintenance of the multipotentiality of a Neural Stem Cell (NSC), said method comprising culturing said NSC as an adherent population on a coated surface in the presence of LIF.

6. The method of claim 5, wherein said NSC adheres to a surface coated with polyornithine and fibronectin.

7. The method of claim 5, wherein said NSC is derived from a human.

8. The method of claim 5, wherein exogenous genetic material has been introduced into said NSC.

9. A method for the in vitro expansion and maintenance of the multipotentiality of an NSC, said method comprising culturing said NSC as an adherent population on a coated surface in the presence of LIF, wherein the expression of MHC class II molecule in said NSC is regulated by said method.

10. A method for the in vitro expansion and maintenance of the multipotentiality of a Neural Stem Cell (NSC), wherein the expression of MHC class II molecule is reduced in said NSC when compared to an otherwise identical NSC cultured in the continuous presence of LIF, said method comprising:
a) culturing said NSC as an adherent population on a coated surface in the presence of LIF for a period of time, then
b) removing LIF from the culture, and
c) culturing said NSC as an adherent population on a coated surface in the absence of LIF for a period of time.

11. The method of claim 10, wherein said NSC is cultured in the presence of LIF for about 7 days.

12. The method of claim 10, wherein said NSC is cultured in the absence of LIF for about 7 days.

13. The method of claim 10, wherein following (c), said NSC exhibits a doubling rate of about 28-36 hours.

14. An isolated Neural Stem Cell (NSC) prepared by a method of culturing said NSC comprising:
a) culturing said NSC as an adherent population on a coated surface in the presence of LIF for a period of time, then
b) removing LIF from the culture, and
c) culturing said NSC as an adherent population on a coated surface in the absence of LIF for a period of time.

15. The isolated NSC of claim 14, wherein said NSC exhibits a doubling rate of about 28-36 hours.

16. The isolated NSC of claim 14, wherein said NSC exhibits a reduced level of MHC class II molecule expression compared to the level of MHC class II molecule expression on an otherwise identical NSC cultured in the continuous presence of LIF.

17. The isolated NSC of claim 14, wherein said NSC is derived from a human.

18. The isolated NSC of claim 14, wherein exogenous genetic material has been introduced into said NSC.

19. A method of treating a human patient having a disease, disorder or condition of the central nervous system, the method comprising:
i) obtaining an isolated Neural Stem Cell (NSC),
ii) culturing said NSC as an adherent population on a coated surface in the presence of LIF for a period of time,
iii) removing LIF from the culture,
iv) culturing said NSC as an adherent population on a coated surface in the absence of LIF for a period of time, and
v) administering said cultured NSC to the central nervous system of said human patient.

20. The method of claim 19, wherein said disease, disorder or condition of the central nervous system is selected from the group consisting of a genetic disease, brain trauma, Huntington's disease, Alzheimer's disease, Parkinson's disease, spinal cord injury, stroke, multiple sclerosis, cancer, CNS lysosomal storage diseases and head trauma, epilepsy.

21. The method of claim 19, wherein said disease, disorder or condition is injury to the tissue or cells of said central nervous system.

22. The method of claim 19, wherein said disease, disorder or condition is a brain tumor.

23. The method of claim 19, wherein said cultured NSC administered to said central nervous system remains present and/or replicates in said central nervous system.

24. The method of claim 19, wherein prior to administering said NSC, said NSC is further cultured in vitro in a differentiation medium.

25. The method of claim 19, wherein prior to administering said NSC, said NSC is genetically modified.

26. A composition comprising an isolated Neural Stem Cell (NSC) and a biologically compatible lattice, wherein said NSC is prepared by a method comprising:
a) culturing said NSC as an adherent population on a biologically compatible lattice in the presence of LIF for a period of time, then
b) removing LIF from the culture, and
c) culturing said NSC as an adherent population on a biologically compatible lattice in the absence of LIF for a period of time.

27. The composition of claim 26, wherein the lattice comprises polymeric material.

28. The composition of claim 26, wherein the polymeric material is formed of polymer fibers as a mesh or sponge.

29. The composition of claim 26, wherein the polymeric material comprises monomers selected from the group of monomers consisting of glycolic acid, lactic acid, propyl fumarate, caprolactone, hyaluronan, hyaluronic acid and combinations thereof.

30. The composition of claim 26, wherein the polymeric material comprises proteins, polysaccharides, polyhydroxy acids, polyorthoesters, polyanhydrides, polyphosphazenes, synthetic polymers or combinations thereof.

31. The composition of claim 26, wherein the polymeric material is a hydrogel formed by crosslinking of a polymer suspension having the cells dispersed therein.

32. The composition of claim 26, wherein said biologically compatible lattice is further coated with polyornithine.

33. The composition of claim 26, wherein said biologically compatible lattice is further coated with fibronectin.

34. The composition of claim 26, wherein said biologically compatible lattice is further coated with polyornithine and fibronectin.

35. A method for the in vitro expansion and maintenance of the multipotentiality of a Neural Stem Cell (NSC), said method comprising:
a) culturing said NSC as an adherent population on a biologically compatible lattice in the presence of LIF for a period of time, then
b) removing LIF from the culture, and
c) culturing said NSC as an adherent population on a biologically compatible lattice in the absence of LIF for a period of time.

36. The method of claim 35, wherein said biologically compatible lattice is further coated with polyornithine.

37. The method of claim 35, wherein said biologically compatible lattice is further coated with fibronectin.

38. The method of claim 35, wherein said biologically compatible lattice is further coated with polyornithine and fibronectin.
